(19) United States
(12) Patent Application Publication
(10) Pub. No.: US 2009/0215115 A1
(43) Pub. Date: Aug. 27, 2009

(54) SIALYLTRANSFERASES COMPRISING CONSERVED SEQUENCE MOTIFS

(75) Inventors: Michel Gilbert, Gatineau (CA); Warren W.. Wakarchuk, Ottawa (CA)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: National Research Council of Canada, Ottawa, ON (CA)

(21) Appl. No.: 11/815,748

(22) PCT Filed: Sep. 16, 2005
(86) PCT No.: PCT/CA05/01432

§ 371 Date: Oct. 1, 2008

Related U.S. Application Data
(60) Provisional application No. 60/610,807, filed on Sep. 17, 2004.

(51) Int. Cl. C12P 21/02; C12N 9/10; C07H 21/00; C12N 15/63; C12N 1/00; C12P 19/00

(52) U.S. Cl. 435/69.1; 435/193; 536/23.2; 435/320.1; 435/243; 435/72

(57) ABSTRACT

The present invention provides, e.g., sialyltransferase proteins comprising conserved sequence motifs, including α-2,3-sialyltransferase proteins from C. jejuni strains O:36 and O:19. The invention also provides methods of making sialylated products using those sialyltransferases.

### CROSS-REFERENCES TO RELATED APPLICATIONS

This application claims the benefit of U.S. Provisional Application No. 60/610,807, filed Sep. 17, 2004, which is herein incorporated by reference for all purposes.

### FIELD OF THE INVENTION

The present invention provides, e.g., sialyltransferase proteins comprising conserved sequence motifs, including α-2,3-sialyltransferase proteins from C. jejuni strains O:36 and O:19. The invention also provides methods of making sialylated products using those sialyltransferases.

### BACKGROUND OF THE INVENTION

Carbohydrates are now recognized as being of major importance in many cell-cell recognition events, notably the adhesion of bacteria and viruses to mammalian cells in pathogenesis and leukocyte-endothelial cell interaction through selectins in inflammation (Varki (1993) Glycobiology 3: 97-130). Moreover, sialylated glycoconjugates that are found in bacteria (Preston et al. (1996) Crit. Rev. Microbiol. 22:139-180; Reuter et al. (1996) Biol. Chem. Hoppe-Seyler 377:325-342) are thought to mimic oligosaccharides found in mammalian glycolipids to evade the host immune response (Moran et al. (1996) FEMS Immunol. Med. Microbiol. 16:105-115). Molecular mimicry of host structures by the saccharide portion of lipopolysaccharide (LPS) is considered to be a virulence factor of various mucosal pathogens, which use this strategy to evade a host immune response (Moran et al. (1996) FEMS Immunol. Med. Microbiol. 16: 105-115; Moran et al. (1996) J. Endotoxin Res. 3: 521-531).

The oligosaccharide structures involved in these and other processes are potential therapeutic agents, but they are time consuming and expensive to make by traditional chemical means. A very promising route to production of specific oligosaccharide structures is through the use of the enzymes which make them in vivo, the glycosyltransferases. Such enzymes can be used as regio- and stereoselective catalysts for the in vitro synthesis of oligosaccharides (Ichikawa et al. (1992) Anal. Biochem. 202: 215-238). Sialyltransferases are a group of glycosyltransferases that transfer sialic acid from an activated sugar nucleotide to acceptor oligosaccharides found on glycoproteins, glycolipids or polysaccharides. The large number of sialylated oligosaccharide structures has led to the characterization of many different sialyltransferases involved in the synthesis of various structures. Sialyltransferases have been isolated and characterized from mammals and other eukaryotes and from microbes, including C. jejuni, Neisseria, Haemophilus, and E. coli. (Tsuji et al. (1996) Glycobiology 6:v-vii; U.S. Pat. Nos. 6,503,744; 6,699,705; 6,096,529; 6,210,933; and Weisgerber et al. (1991) Glycobiol. 1:357-365).

Large scale enzymatic synthesis of oligosaccharides depends on the availability of sufficient quantities of the required glycosyltransferases. However, production of glycosyltransferases in sufficient quantities for use in preparing oligosaccharide structures has been problematic. Expression of many mammalian glycosyltransferases has been achieved involving expression in eukaryotic hosts which can involve expensive tissue culture media and only moderate yields of protein (Kleene et al. (1994) Biochem. Biophys. Res. Commun. 201: 160-167; Williams et al. (1995) Glycoconjugate J. 12: 755-761). Expression in E. coli has been achieved for mammalian glycosyltransferases, but these attempts have produced mainly insoluble forms of the enzyme from which it has been difficult to recover active enzyme in large amounts (Aoki et al. (1990) EMBO. J. 9:3171-3178; Nishiu et al. (1995) Biosci. Biotech. Biochem. 59 (9): 1750-1752). Furthermore, because of the biological activity of their products, mammalian sialyltransferases generally act in specific tissues, cell compartments and/or developmental stages to create precise sialyloglycans.

Mammalian sialytransferases commonly share a conserved sialyltransferase binding motif that aids in identification of the enzymes. (Datta and Paulson, J. Biol. Chem. 270:1497-1500 (1995). This mammalian motif appears to not be conserved in bacterial enzymes. (See, e.g., Chiu et al., Nat. Struct. Mol. Biol. 11: 163-70 (2004) Epub 2004 Jan. 18.) Because identification of additional bacterial sialyltransferases would aid in, e.g., synthesis of desired oligosaccharides with biological activity, identification and characterization of new bacterial sialyltransferases would thus be useful in the development of these technologies. The present invention fulfills this and other needs.

### BRIEF SUMMARY OF THE INVENTION

This disclosure provides description of newly recognized amino acid motifs that can be used to identify sialyltransferase polypeptides. The sialyltraferase polypeptides are members of a genus of proteins that transfer sialic acid from a donor substrate to an acceptor substrate; that comprises a sialyltransferase motif A and a sialyltransferase motif B as defined herein; the following known sialyltransferase polypeptides (identified by accession number of amino acid or an encoding nucleic acid) are not included in the claimed genus: GenBank AF130466, GenBank AX934425, GenBank AX934434, GenBank AX934427, GenBank AX934431, GenBank AF401529, GenBank AX934436, GenBank AX934429, GenBank AY044156, GenBank AF400047, GenBank AY297047, GenBank AF305571, GenBank AL139077, GenBank X57315, and GenBank AE006157 Also excluded from the genus is the artificially derived sialyltransferase protein consensus sequence derived from CD: pfam06002.2, CST-I, the conserved data bases domain shown in FIG. 4. In some embodiments the sialyltransferase motif A is DVFRCNQFYFED/E (SEQ ID NO: 1), i.e., DVFRCNQFYFED (SEQ ID NO:3) or DVFRCNQFYFEE (SEQ ID NO:4). In further embodiments, the sialyltransferase motif A is DVFRCNQFYFED/E (SEQ ID NO: 1) and the sialyltransferase motif B is RITSGVYMC (SEQ ID NO:2). In other embodiments, the sialyltransferase motif B is RITSGVYMC (SEQ ID NO:2).

Sialyltransferase polypeptides comprising sialyltransferase motif A and a sialyltransferase motif B can have α-2,3-sialyltransferase activity, α-2,8-sialyltransferase activity, or can have dual α-2,3/8-sialyltransferase activity.

Sialyltransferase polypeptides comprising sialyltransferase motif A and a sialyltransferase motif B can transfer a sialic acid moiety from a donor molecule to an acceptor molecule, e.g., oligosaccharide, a glycolipid, a glycopeptide, or a glycoprotein.

In some embodiments, a sialyltransferase polypeptide comprising sialyltransferase motif A and a sialyltransferase motif B is truncated and retains activity. In some embodiments, a sialyltransferase polypeptide comprising sialyltransferase motif A and a sialyltransferase motif B is a bacterial protein. A bacterial sialyltransferase polypeptide comprising sialyltransferase motif A and a sialyltransferase motif B can be derived originally from a member of the family Vibrionaceae. In other embodiments, the bacterial sialyltransferase polypeptide comprising sialyltransferase motif A and a sialyltransferase motif B can be derived originally from Haemophilus influenzae, Pasteurella multocida, or Campylobacter species. In some embodiments, the bacterial sialyltransferase polypeptide comprising sialyltransferase motif A and a sialyltransferase motif B can be derived originally from Campylobacter jejuni, e.g., strain O:19 or strain O:36.

Sialyltransferase polypeptides comprising sialyltransferase motif A and a sialyltransferase motif B can include an amino acid tag or can be fused to an accessory enzyme.

In another aspect this disclosure provides isolated or recombinant sialyltransferase polypeptide that transfers sialic acid from a donor substrate to an acceptor substrate and that includes an amino acid sequence with at least 98% identity to the amino acid sequence of FIG. 2 (O:36 amino acid sequence, SEQ ID NO:6). The sialyltransferase polypeptide has α-2,3-sialyltransferase activity in some embodiments. In some embodiments, the sialyltransferase polypeptide uses an oligosaccharide, a glycolipid, a glycopeptide, or a glycoprotein as an acceptor molecule. The sialyltransferase polypeptide can include an amino acid tag or can be fused to an accessory enzyme. In a further embodiment, the amino acid sequence of FIG. 2 (O:36 amino acid sequence, SEQ ID NO:6) and the amino acid sequence of FIG. 3 (O:19 amino acid sequence, SEQ ID NO:8).

In one embodiment, this disclosure provides an isolated or recombinant sialyltransferase polypeptide that transfers sialic acid from a donor substrate to an acceptor substrate and that comprises amino acids 1-283 of the amino acid sequence of FIG. 2 (O:36 amino acid sequence, SEQ ID NO:6). In a further embodiment, the isolated or recombinant sialyltransferase polypeptide comprises amino acids 1-285 of the amino acid sequence of FIG. 2 (O:36 amino acid sequence, SEQ ID NO:6).

In another embodiment, this disclosure provides an isolated or recombinant sialyltransferase polypeptide that transfers sialic acid from a donor substrate to an acceptor substrate and that comprises amino acids 1-285 of the amino acid sequence of FIG. 3 (O:19 amino acid sequence, SEQ ID NO:8). In a further embodiment, the isolated or recombinant sialyltransferase polypeptide comprises amino acids 1-293 of the amino acid sequence of FIG. 3 (O:19 amino acid sequence, SEQ ID NO:8).

This disclosure also provides nucleic acids that encode isolated or recombinant sialyltransferase polypeptides that transfer sialic acid from a donor substrate to an acceptor substrate, e.g., an isolated or recombinant nucleic acid that comprises a sialyltransferase polynucleotide sequence that comprises a nucleotide sequence with at least 98% identity to the nucleic acid sequence of FIG. 2 (O:36 nucleic acid sequence, SEQ ID NO:5). The encoded sialyltransferase polypeptide transfers sialic acid to acceptor molecules including, e.g., oligosaccharides, glycolipids, glycopeptides, and glycoproteins. The encoded sialyltransferase polypeptide can also include an amino acid tag; and in some embodiments is fused to an accessory enzyme to form a fusion protein. In additional embodiments, the sialyltransferase polynucleotide sequence comprises either the nucleic acid sequence of FIG. 2 (O:36 nucleic acid sequence, SEQ ID NO:5) or the nucleic acid sequence of FIG. 3 (O:19 nucleic acid sequence, SEQ ID NO:7). Additional embodiments of sialyltransferase polynucleotide sequences included e.g., nucleotides 1-849 of FIG. 2 (O:36 nucleic acid sequence, SEQ ID NO:5), nucleotides 1-855 of FIG. 2 (O:36 nucleic acid sequence, SEQ ID NO:5), nucleotides 1-855 of FIG. 3 (O:19 nucleic acid sequence, SEQ ID NO:7), and nucleotides 1-888 of FIG. 3 (O:19 nucleic acid sequence, SEQ ID NO:7). Further embodiments include polypeptides that comprise amino acid sequences of the Lic3A and Lic3A2 sialyltransferase proteins from H. influenzae, e.g., the amino acid sequences of FIGS. 5 and 6 or amino acids sequences with greater than 80%, 85%, 90%, 91%, 92%, 93%, 94%, 95%, 96%, 97%, 98%, 99% identity to the amino acid sequences of FIGS. 5 and 6.

This disclosure also provides nucleic acids that encode isolated or recombinant sialyltransferase polypeptides that transfer sialic acid from a donor substrate to an acceptor substrate, e.g., a sialyltransferase polynucleotide sequence that encodes a sialyltransferase polypeptide that comprises amino acids 1-285 of the amino acid sequence of FIG. 2 (O:36 amino acid sequence, SEQ ID NO:6), or a sialyltransferase polynucleotide sequence that encodes a sialyltransferase polypeptide that comprises amino acids 1-285 of the amino acid sequence of FIG. 3 (O:19 amino acid sequence, SEQ ID NO:8), or a sialyltransferase polynucleotide sequence that encodes a sialyltransferase polypeptide that comprises amino acids 1-293 of the amino acid sequence of FIG. 3 (O:19 amino acid sequence, SEQ ID NO:8). Also included are nucleic acids that encode the Lic3A and Lic3A2 sialyltransferase proteins from H. influenzae, e.g., the amino acid sequences of FIGS. 5 and 6 or amino acids sequences with greater than 80%, 85%, 90%, 91%, 92%, 93%, 94%, 95%, 96%, 97%, 98%, 99% identity to the amino acid sequences of FIGS. 5 and 6.

In a further aspect this disclosure provides expression vectors the comprise sialyltransferase polynucleotide sequences; host cells that comprises the expression vectors, and methods of making the sialyltransferase polypeptides described herein, by growing the host cells under conditions suitable for expression of the sialyltransferase polypeptide.

Another aspect of this disclosure provides methods of producing sialylated product saccharides by contacting an acceptor substrate with a donor substrate comprising a sialic acid moiety and a sialyltransferase polypeptide comprising sialytransferases motifs A and B; and allowing transfer of a sialic acid moiety to the acceptor saccharide to occur, thereby producing the sialylated product saccharide.

### BRIEF DESCRIPTION OF THE DRAWINGS

FIG. 1 provides an alignment of known sialyltransferases and two previously unknown sialytransferases (Cst-I O:19 and Cst-I O:36) and demonstrates the conserved nature of amino acid motif A and amino acid motif B. The alignment of the 18 protein sequences was performed using CLUSTAL-W. The * indicate residues that are conserved in all 18 sequences. The residues in motifs A and B are underlined and in bold. Notice that the last residue of motif A is conserved in all sequences except for PM1174 from Pasteurella multocida. Residues 1-300 were included for Cst-I OH4384, Cst-I O:19 and Cst-I O:36; additional C-terminal residues were omitted. The other sequences are full length. A consensus sequence (Prim. cons.), based on the alignment is shown in the bottom row.

FIG. 2 provides a nucleic acid sequence and an amino acid sequence for Cst-I from Campylobacter jejuni strain O:36.

FIG. 3 provides a nucleic acid sequence and an amino acid sequence for Cst-I from Campylobacter jejuni strain O:19.

FIG. 4 provides the consensus sequence of a sialyltransferase protein derived from CD: pfam06002.2, CST-I, the conserved data bases domain.

FIG. 5 provides a nucleic acid sequence and an amino acid sequence for a sialyltransferase of the invention, the lic3A nucleic acid and protein from Haemophilus influenzae 86-028NP.

FIG. 6 provides a nucleic acid sequence and an amino acid sequence for a sialyltransferase of the invention, the lic3A2 nucleic acid and protein from Haemophilus influenzae 86-028NP.

### DETAILED DESCRIPTION OF THE INVENTION

### I. Introduction

The present invention provides amino acid sequences of conserved bacterial sialyltransferase motifs A and B, that can be used to identify bacterial sialyltransferase polypeptides that comprise the conserved motifs. Novel sialyltransferases that comprise the conserved sialyltransferase motifs can be used to sialylate e.g., oligosaccharides, glycopeptides or glycoproteins, or glycolipids. The invention also provides the amino acid and nucleic acid sequences of novel sialyltransferases, e.g., Cst-I proteins from C. jejuni strains O:19 and O:36 and lic3A and lic3A2 sialyltransferases from Haemophilus influenzae.

### II. Definitions

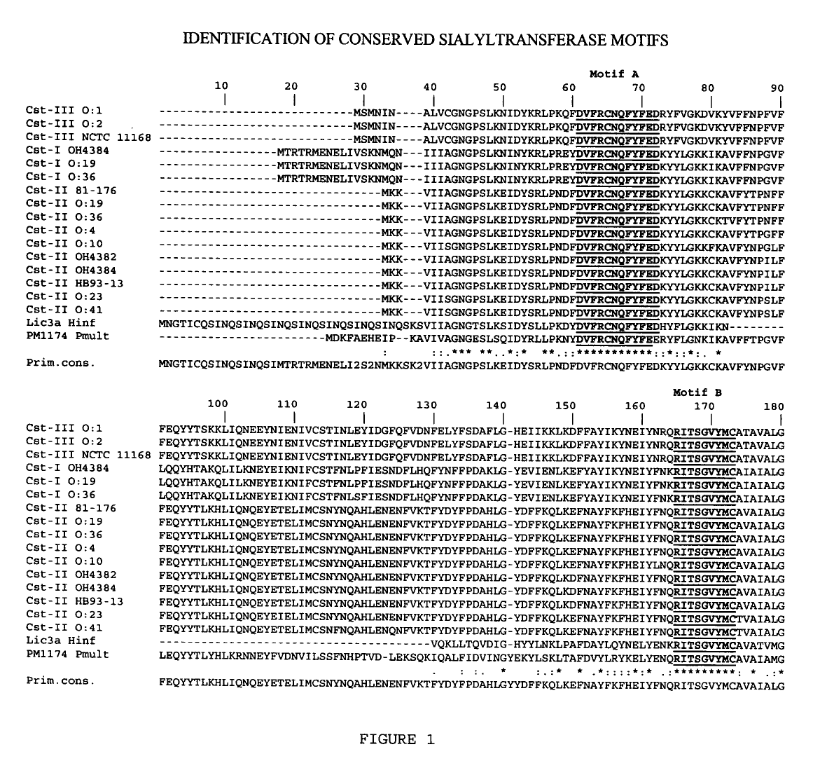

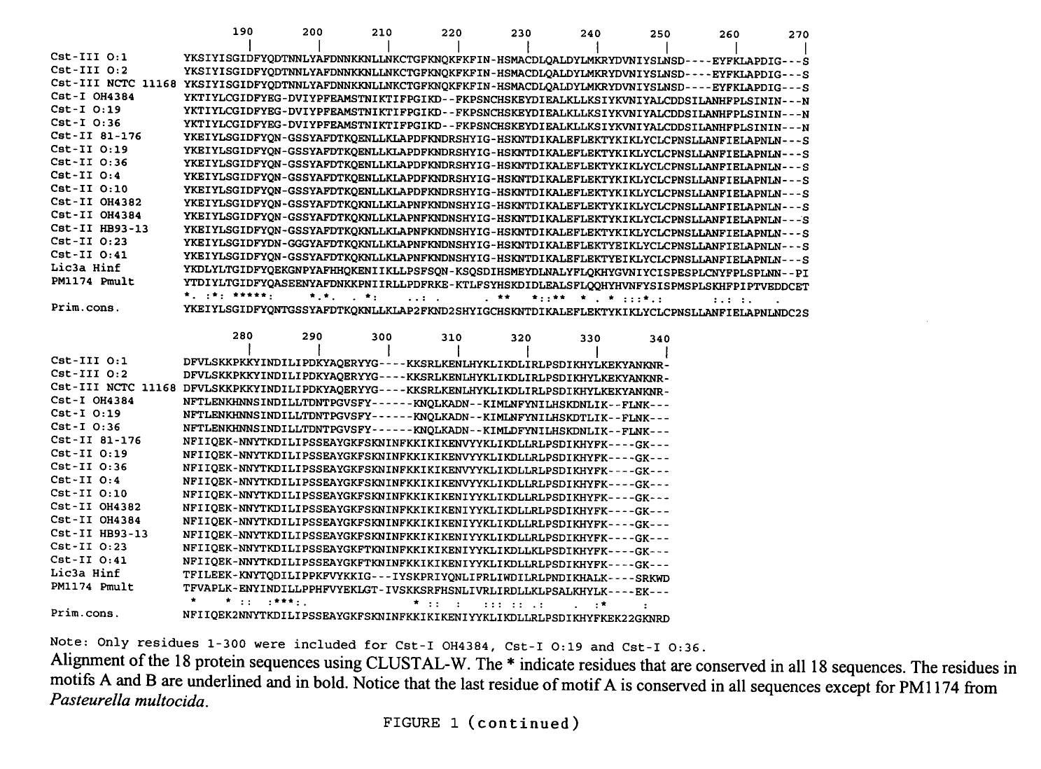

As used herein “sialyltransferase polypeptide” refers to a polypeptide that comprises two conserved motifs, sialyltransferase motif A and sialyltransferase motif B, described below, and that has sialytransferase activity, i.e., the protein catalyzes the transfer of a donor substrate, such as an activated sialic acid molecule, to an acceptor substrate, such as an oligosaccharide, glycolipid, or glycoprotein. The identification of the conserved motifs is based on sequence comparison of 11 known sialyltransferase proteins, see, e.g., FIG. 1, and on the position of the conserved residues at a substrate binding site of a sialyltransferase protein, e.g., the conserved residues appear to function as components of a substrate binding site. (See, e.g., Chiu et al., Nat. Struct. Mol. Biol. 11: 163-70 (2004) Epub 2004 Jan. 18.) This group of sialyltransferase polypeptides includes proteins that catalyze addition of the sialic acid residue in an α2,3 linkage, proteins that catalyze addition of the sialic acid residue in an α2,8 linkage, and dual function proteins that catalyze addition of the sialic acid residue in an α2,3 linkage and an α2,8 linkage. Sialyltransferases that catalyze addition of a sialic acid residue in other linkages, e.g., α2,6 linkage are also included in the group.

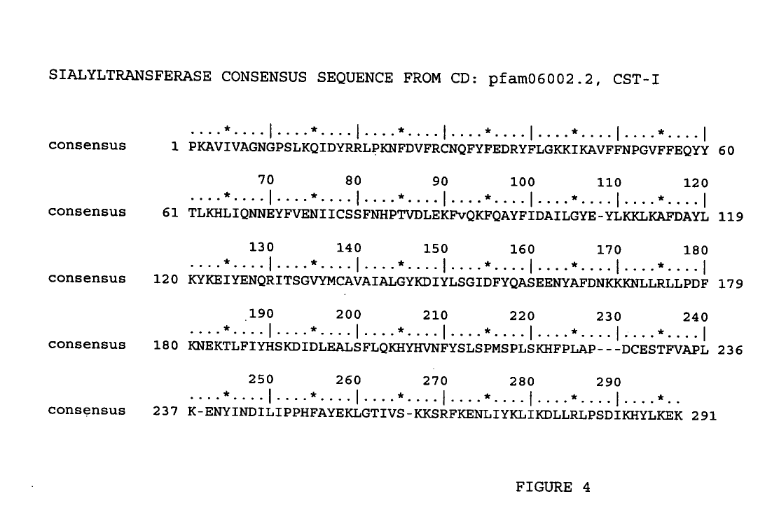

In some embodiments, sialyltransferase polypeptides are from microorganisms, in further embodiments the sialyltransferase polypeptides are from bacteria. Some of the bacteria that have the disclosed sialyltransferases include Campylobacter, Haemophilus, and Pasteurella. Campylobacter jejuni is known to have three classes of sialyltransferases, i.e., Cst-I, Cst-II, and Cst-III. Members of each of the three C. jejuni classes of sialytransferases are included in the sialyltransferase polypeptides of the invention. Sialyltransferase protein or polypeptide, as defined herein, does not include the sialyltransferase proteins disclosed in the following accession numbers: GenBank AAF13495; GenBank AX934425; GenBank AX934434; GenBank AX934427; GenBank AX934431; GenBank AAL06004; GenBank AX934436; GenBank AX934429; GenBank AAK73183; GenBank AAK85419; the sialyltransferase encoded by GenBank AY297047, shown as Cst-II HB93-13 in FIG. 1; GenBank AAL09368; GenBank NP_{—}282288; GenBank CAA40567; or GenBank AAK03258. The definition of sialyltransferases also excludes the artificially derived sialyltransferase protein consensus sequence derived from CD: pfam06002.2, CST-I, the conserved data bases domain shown in FIG. 4. Other sialyltransferases sequences excluded from the genus are Campylobacter sialyltransferases disclosed in U.S. Pat. No. 6,503,744 issued Jan. 7, 2003 and U.S. Pat. No. 6,699,705 issued Mar. 2, 2004, both of which are herein incorporated by reference; and sequences disclosed in the following accession numbers: CAA40567, CAB73395, AAL09368, AAL36462, ZP_{—}00322176, ZP_{—}00321441, ZP_{—}00155359, ZP_{—}00156191, AAL05990, AAG43979, AAK03258, AAF13495, AAK96001, AAK91725, AAL06004, CAB73395, AAL09368, NP_{—}245125, and AAL36462.

As used herein “sialyltransferase motif A” refers to an amino acid sequence found in sialyltransferase polypeptides, i.e., DVFRCNQFYFED/E, (SEQ ID NO: 1), and conservatively modified variants of that sequence. Thus, sialyltransferase motif A refers to DVFRCNQFYFED, (SEQ ID NO:3), and DVFRCNQFYFEE, (SEQ ID NO:4), and conservatively modified variants of those sequences, as well. As used herein “sialyltransferase motif B” refers to an amino acid sequence found in sialyltransferase polypeptides, i.e., RITSGVYMC, (SEQ ID NO:2), and conservatively modified variants of that sequence. In general sialyltransferase motif A is found amino terminal relative to sialyltransferase B in a sialyltransferase polypeptide. Spacing between the two sialyltransferase motifs is not critical. In some embodiments, about 30, 35, 40, 44, 45, 50, 55, 60, 65, 70, 75, 80, 85, 86, 87, 88, 89, 90, 91, 92, 93, 94, 95, 96, 97, 98, 99, 100, 105, or 110 amino acid residues separate the two motifs. Typically, spacing between the two motifs is between e.g., 80 and 100 residues or between 90 and 95 residues, and for some embodiments is usually, e.g., 91, 92, or 93 amino acid residues.

As used herein, a “truncated sialyltransferase polypeptide” or grammatical variants, refers to a sialyltransferase polypeptide that has been manipulated to remove at least one amino acid residue, relative to a wild type sialytransferase polypeptide that occurs in nature, so long as the truncated sialyltransferase polypeptide retains enzymatic activity. For example, C. jejuni Cst-I polypeptides comprising amino acids 1 though about 285 are active; C. jejuni Cst-II polypeptides comprising amino acids 1 though about 255 are active; and C. jejuni Cst-III polypeptides comprising amino acids 1 though about 255 are active.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

Those of skill recognize that many amino acids can be substituted for one another in a protein without affecting the function of the protein, i.e., a conservative substitution can be the basis of a conservatively modified variant of a protein such as the disclosed sialyltransferases. An incomplete list of conservative amino acid substitutions follows. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V), Alanine (A); 6) Phenylalanine (F), Tyrosine (Y), Tryptophan (W); 7) Serine (S), Threonine (T), Cysteine (C); and 8) Cysteine (C), Methionine (M) (see, e.g., Creighton, Proteins (1984)).

The cells and methods of the invention are useful for producing a sialylated product, generally by transferring a sialic acid moiety from a donor substrate to an acceptor molecule. The cells and methods of the invention are also useful for producing a sialylated product sugar comprising additional sugar residues, generally by transferring a additional monosaccharide or a sulfate groups from a donor substrate to an acceptor molecule. The addition generally takes place at the non-reducing end of an oligosaccharide, polysaccharide (e.g., heparin, carragenin, and the like) or a carbohydrate moiety on a glycolipid or glycoprotein, e.g., a biomolecule. Biomolecules as defined here include but are not limited to biologically significant molecules such as carbohydrates, oligosaccharides, proteins (e.g., glycoproteins), and lipids (e.g., glycolipids, phospholipids, sphingolipids and gangliosides).

The following abbreviations are used herein:
Ara=arabinosyl;
Fru=fructosyl;
Fuc=fucosyl;
Gal=galactosyl;
GalNAc=N-acetylgalactosaminyl;
Glc=glucosyl;
GlcNAc=N-acetylglucosaminyl;
Man=mannosyl; and
NeuAc=sialyl (N-acetylneuraminyl).

The term “sialic acid” or “sialic acid moiety” refers to any member of a family of nine-carbon carboxylated sugars. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al., J. Biol. Chem. 265: 21811-21819 (1990)). Also included are 9-substituted sialic acids such as a 9-O—C₁-C₆ acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see, e.g., Varki, Glycobiology 2: 25-40 (1992); Sialic Acids: Chemistry, Metabolism and Function, R. Schauer, Ed. (Springer-Verlag, New York (1992)). The synthesis and use of sialic acid compounds in a sialylation procedure is disclosed in international application WO 92/16640, published Oct. 1, 1992.

A “sialylated product saccharide” refers an oligosaccharide, polysaccharide (e.g., heparin, carragenin, and the like) or a carbohydrate moiety, either unconjugated or conjugated to a glycolipid or glycoprotein, e.g., a biomolecule, that includes a sialic acid moiety. Any of the above sialic acid moieties can be used as well as PEGylated sialic acid derivatives. In some embodiments other sugar moieties, e.g., fucose, galactose, glucose, GalNAc, or GluNAc, are also added to the acceptor substrate to produce the sialylated product saccharide. Examples of sialylated product saccharides include, e.g., sialylactose.

The term “PEG” refers to poly(ethylene glycol). PEG is an exemplary polymer that has been conjugated to peptides. The use of PEG to derivatize peptide therapeutics has been demonstrated to reduce the immunogenicity of the peptides and prolong the clearance time from the circulation. For example, U.S. Pat. No. 4,179,337 (Davis et al.) concerns non-immunogenic peptides, such as enzymes and peptide hormones coupled to polyethylene glycol (PEG) or polypropylene glycol. Between 10 and 100 moles of polymer are used per mole peptide and at least 15% of the physiological activity is maintained.

An “acceptor substrate” or an “acceptor saccharide” for a glycosyltransferase, e.g., a sialyltransferase, is an oligosaccharide moiety that can act as an acceptor for a particular glycosyltransferase. When the acceptor substrate is contacted with the corresponding glycosyltransferase and sugar donor substrate, and other necessary reaction mixture components, and the reaction mixture is incubated for a sufficient period of time, the glycosyltransferase transfers sugar residues from the sugar donor substrate to the acceptor substrate. The acceptor substrate can vary for different types of a particular glycosyltransferase. Accordingly, the term “acceptor substrate” is taken in context with the particular glycosyltransferase of interest for a particular application. Acceptor substrates for sialyltransferases and additional glycosyltransferases, are described herein.

A “donor substrate” for glycosyltransferases is an activated nucleotide sugar. Such activated sugars generally consist of uridine, guanosine, and cytidine monophosphate derivatives of the sugars (UMP, GMP and CMP, respectively) or diphosphate derivatives of the sugars (UDP, GDP and CDP, respectively) in which the nucleoside monophosphate or diphosphate serves as a leaving group. For example, a donor substrate for fucosyltransferases is GDP-fucose. Donor substrates for sialyltransferases, for example, are activated sugar nucleotides comprising the desired sialic acid. For instance, in the case of NeuAc, the activated sugar is CMP-NeuAc. Bacterial, plant, and fungal systems can sometimes use other activated nucleotide sugars.

Oligosaccharides are considered to have a reducing end and a non-reducing end, whether or not the saccharide at the reducing end is in fact a reducing sugar. In accordance with accepted nomenclature, oligosaccharides are depicted herein with the non-reducing end on the left and the reducing end on the right. All oligosaccharides described herein are described with the name or abbreviation for the non-reducing saccharide (e.g., Gal), followed by the configuration of the glycosidic bond (α or β), the ring bond, the ring position of the reducing saccharide involved in the bond, and then the name or abbreviation of the reducing saccharide (e.g., GlcNAc). The linkage between two sugars may be expressed, for example, as 2, 3, 2→3, or (2,3). Each saccharide is a pyranose or furanose.

The term “contacting” is used herein interchangeably with the following: combined with, added to, mixed with, passed over, incubated with, flowed over, etc.

Much of the nomenclature and general laboratory procedures required in this application can be found in Sambrook, et al., Molecular Cloning: A Laboratory Manual (2nd Ed.), Vol. 1-3, Cold Spring Harbor Laboratory, Cold Spring Harbor, N.Y., 1989. The manual is hereinafter referred to as “Sambrook et al.”

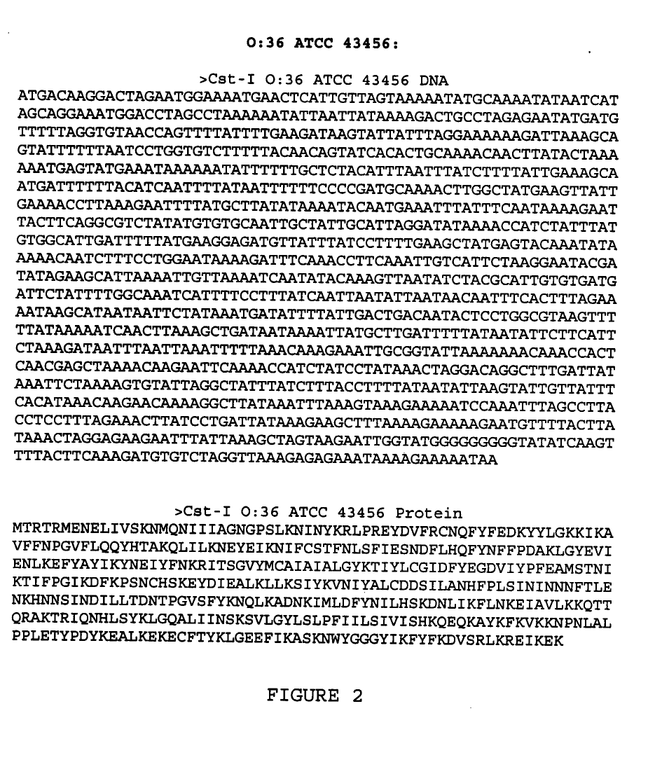

The terms “Cst-I from C. jejuni strain O:36” or a nucleic acid encoding “Cst-I from C. jejuni strain O:36” refer to nucleic acids and polypeptide polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to an amino acid sequence encoded by a Cst-I from C. jejuni strain O:36 nucleic acid (for a Cst-I from C. jejuni strain O:36 nucleic acid sequence, see, e.g., FIG. 2, SEQ ID NO:5) or to an amino acid sequence of a Cst-I from C. jejuni strain O:36 protein (for a Cst-I from C. jejuni strain O:36 protein sequence, see, e.g., FIG. 2, SEQ ID NO:6); (2) bind to antibodies, e.g., polyclonal antibodies, raised against an immunogen comprising an amino acid sequence of a Cst-I from C. jejuni strain O:36 protein, and conservatively modified variants thereof; (3) specifically hybridize under stringent hybridization conditions to an anti-sense strand corresponding to a nucleic acid sequence encoding a Cst-I from C. jejuni strain O:36 protein, and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a Cst-I from C. jejuni strain O:36 nucleic acid or a nucleic acid encoding the catalytic domain. Preferably the catalytic domain has greater than 96%, 97%, 98%, or 99% amino acid identity to the Cst-I from C. jejuni strain O:36 catalytic domain of SEQ ID NO:6. A polynucleotide or polypeptide sequence is typically from a bacteria including, but not limited to, Campylobacter, Haemophilus, and Pasteurella. The nucleic acids and proteins of the invention include both naturally occurring or recombinant molecules. A Cst-I from C. jejuni strain O:36 protein typically has sialyltransferase activity. Sialyltransferase assays can be performed according to methods known to those of skill in the art, using appropriate donor substrates and acceptor substrates, as described herein.

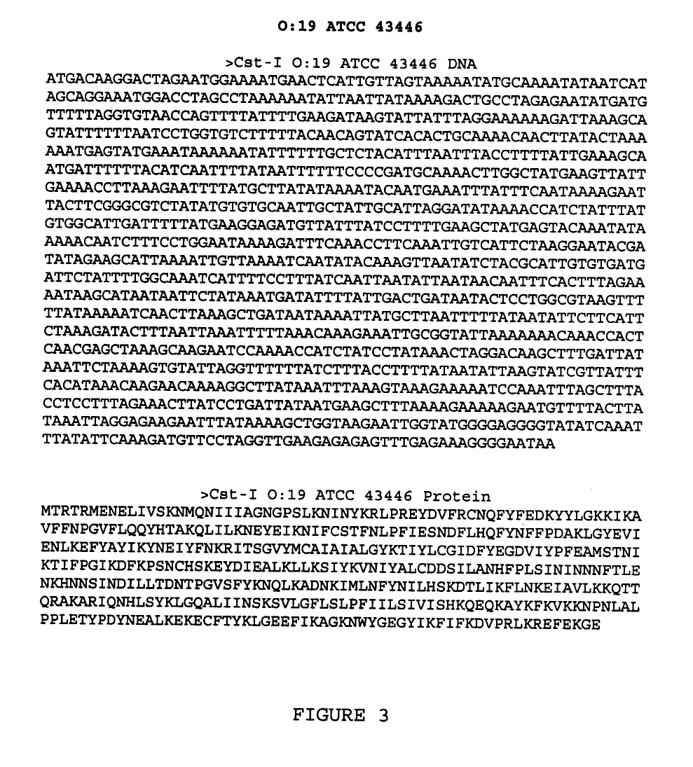

The terms “Cst-I from C. jejuni strain O:19” or a nucleic acid encoding “Cst-I from C. jejuni strain O:19” refer to nucleic acids and polypeptide polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to an amino acid sequence encoded by a Cst-I from C. jejuni strain O:19 nucleic acid (for a Cst-I from C. jejuni strain O:19 nucleic acid sequence, see, e.g., FIG. 3, SEQ ID NO:7) or to an amino acid sequence of a Cst-I from C. jejuni strain O:19 protein (for a Cst-I from C. jejuni strain O:19 protein sequence, see, e.g., FIG. 3, SEQ ID NO:8); (2) bind to antibodies, e.g., polyclonal antibodies, raised against an immunogen comprising an amino acid sequence of a Cst-I from C. jejuni strain O:19 protein, and conservatively modified variants thereof; (3) specifically hybridize under stringent hybridization conditions to an anti-sense strand corresponding to a nucleic acid sequence encoding a Cst-I from C. jejuni strain O:19 protein, and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a Cst-I from C. jejuni strain O:19 nucleic acid or a nucleic acid encoding the catalytic domain. Preferably the catalytic domain has greater than 96%, 97%, 98%, or 99% amino acid identity to the Cst-I from C. jejuni strain O:19 catalytic domain of SEQ ID NO:8. A polynucleotide or polypeptide sequence is typically from a bacteria including, but not limited to, Campylobacter, Haemophilus, and Pasteurella. The nucleic acids and proteins of the invention include both naturally occurring or recombinant molecules. A Cst-I from C. jejuni strain O:19 protein typically has sialyltransferase activity. Sialyltransferase assays can be performed according to methods known to those of skill in the art, using appropriate donor substrates and acceptor substrates, as described herein.

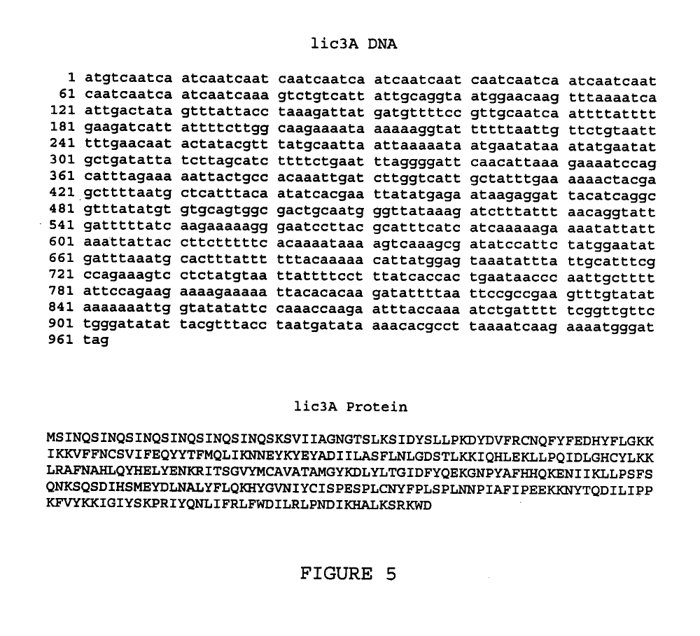

The terms “lic3A sialyltransferase from H. influenzae” or a nucleic acid encoding “lic3A sialyltransferase from H. influenzae” refer to nucleic acids and polypeptide polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to an amino acid sequence encoded by a lic3A sialyltransferase nucleic acid from H. influenzae (for a lic3A sialyltransferase nucleic acid sequence, see, e.g., FIG. 5) or to an amino acid sequence of a lic3A sialyltransferase polypeptide from H. influenzae (for a lic3A sialyltransferase amino acid sequence, see, e.g., FIG. 5,); (2) bind to antibodies, e.g., polyclonal antibodies, raised against an immunogen comprising an amino acid sequence of a lic3A sialyltransferase protein, and conservatively modified variants thereof; (3) specifically hybridize under stringent hybridization conditions to an anti-sense strand corresponding to a nucleic acid sequence encoding a lic3A sialyltransferase protein, and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a lic3A sialyltransferase nucleic acid sequence or a nucleic acid encoding the catalytic domain of a lic3A sialyltransferase protein. Preferably the catalytic domain has greater than 96%, 97%, 98%, or 99% amino acid identity to the lic3A sialyltransferase catalytic domain. A polynucleotide or polypeptide sequence is typically from a bacteria including, but not limited to, Campylobacter, Haemophilus, and Pasteurella. The nucleic acids and proteins of the invention include both naturally occurring or recombinant molecules. A lic3A sialyltransferase from H. influenzae typically has sialyltransferase activity. Sialyltransferase assays can be performed according to methods known to those of skill in the art, using appropriate donor substrates and acceptor substrates, as described herein. Lic3A proteins are disclosed at Accession number CP000057 and at Munson et al., J. Bacteriol. 187:4627-4636 (2005).

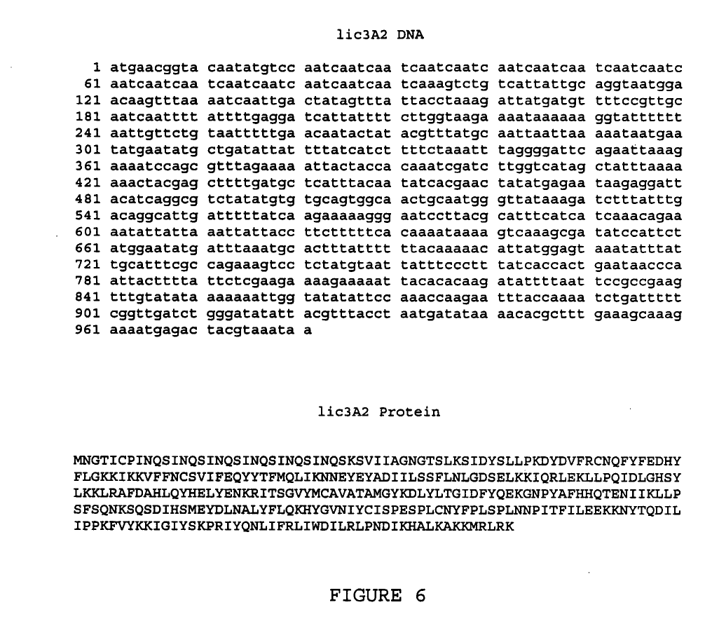

The terms “lic3A2 sialyltransferase from H. influenzae” or a nucleic acid encoding “lic3A2 sialyltransferase from H. influenzae” refer to nucleic acids and polypeptide polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to an amino acid sequence encoded by a lic3A2 sialyltransferase nucleic acid from H. influenzae (for a lic3A2 sialyltransferase nucleic acid sequence, see, e.g., FIG. 6) or to an amino acid sequence of a lic3A2 sialyltransferase polypeptide from H. influenzae (for a lic3A2 sialyltransferase amino acid sequence, see, e.g., FIG. 6); (2) bind to antibodies, e.g., polyclonal antibodies, raised against an immunogen comprising an amino acid sequence of a lic3A2 sialyltransferase protein, and conservatively modified variants thereof; (3) specifically hybridize under stringent hybridization conditions to an anti-sense strand corresponding to a nucleic acid sequence encoding a lic3A2 sialyltransferase protein, and conservatively modified variants thereof, (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a lic3A2 sialyltransferase nucleic acid sequence or a nucleic acid encoding the catalytic domain of a lic3A2 sialyltransferase protein. Preferably the catalytic domain has greater than 96%, 97%, 98%, or 99% amino acid identity to the lic3A2 sialyltransferase catalytic domain. A polynucleotide or polypeptide sequence is typically from a bacteria including, but not limited to, Campylobacter, Haemophilus, and Pasteurella. The nucleic acids and proteins of the invention include both naturally occurring or recombinant molecules. A lic3A2 sialyltransferase from H. influenzae typically has sialyltransferase activity. Sialyltransferase assays can be performed according to methods known to those of skill in the art, using appropriate donor substrates and acceptor substrates, as described herein. Lic3A2 proteins are disclosed at Accession number CP000057.1 and at Munson et al., J. Bacteriol. 187:4627-4636 (2005).

“Commercial scale” refers to gram scale production of a sialylated product in a single reaction. In preferred embodiments, commercial scale refers to production of greater than about 50, 75, 80, 90, 100, 125, 150, 175, or 200 grams of sialylated product.

The recombinant proteins of the invention can be constructed and expressed as a fusion protein with a molecular “purification tag” at one end, which facilitates purification or identification of the protein. Such tags can also be used for immobilization of a protein of interest during the glycosylation reaction. Suitable tags include “epitope tags,” which are a protein sequence that is specifically recognized by an antibody. Epitope tags are generally incorporated into fusion proteins to enable the use of a readily available antibody to unambiguously detect or isolate the fusion protein. A “FLAG tag” is a commonly used epitope tag, specifically recognized by a monoclonal anti-FLAG antibody, consisting of the sequence AspTyrLysAspAspAsp AspLys or a substantially identical variant thereof. Other suitable tags are known to those of skill in the art, and include, for example, an affinity tag such as a hexahistidine peptide, which will bind to metal ions such as nickel or cobalt ions or a myc tag. Proteins comprising purification tags can be purified using a binding partner that binds the purification tag, e.g., antibodies to the purification tag, nickel or cobalt ions or resins, and amylose, maltose, or a cyclodextrin. Purification tags also include maltose binding domains and starch binding domains. Purification of maltose binding domain proteins is known to those of skill in the art. Starch binding domains are described in WO 99/15636, herein incorporated by reference. Affinity purification of a fusion protein comprising a starch binding domain using a betacylodextrin (BCD)-derivatized resin is described in U.S. Ser. No. 60/468,374, filed May 5, 2003, herein incorporated by reference in its entirety.

The term “nucleic acid” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. Unless otherwise indicated, a particular nucleic acid sequence includes the complementary sequence thereof. The terms nucleic acid, “nucleic acid sequence”, and “polynucleotide” are used interchangeably herein.

The term “operably linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant nucleic acid” refers to a nucleic acid that was artificially constructed (e.g., formed by linking two naturally-occurring or synthetic nucleic acid fragments). This term also applies to nucleic acids that are produced by replication or transcription of a nucleic acid that was artificially constructed. A “recombinant polypeptide” is expressed by transcription of a recombinant nucleic acid (i.e., a nucleic acid that is not native to the cell or that has been modified from its naturally occurring form), followed by translation of the resulting transcript.

A “heterologous polynucleotide” or a “heterologous nucleic acid”, as used herein, is one that originates from a source foreign to the particular host cell, or, if from the same source, is modified from its original form. Thus, a heterologous glycosyltransferase gene in a prokaryotic host cell includes a glycosyltransferase gene that is endogenous to the particular host cell but has been modified. Modification of the heterologous sequence may occur, e.g., by treating the DNA with a restriction enzyme to generate a DNA fragment that is capable of being operably linked to a promoter. Techniques such as site-directed mutagenesis are also useful for modifying a heterologous sequence.

A “subsequence” refers to a sequence of nucleic acids or amino acids that comprise a part of a longer sequence of nucleic acids or amino acids (e.g., polypeptide) respectively.

A “recombinant expression cassette” or simply an “expression cassette” is a nucleic acid construct, generated recombinantly or synthetically, with nucleic acid elements that are capable of affecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

A “fusion sialyltransferase polypeptide” or a “fusion glycosyltransferase polypeptide” of the invention is a polypeptide that contains a glycosyltransferase catalytic domain and a second catalytic domain from an accessory enzyme (e.g., a CMP-Neu5Ac synthetase). The fusion polypeptide is capable of catalyzing the synthesis of a sugar nucleotide (e.g., CMP-NeuAc) as well as the transfer of the sugar residue from the sugar nucleotide to an acceptor molecule. Typically, the catalytic domains of the fusion polypeptides will be at least substantially identical to those of glycosyltransferases and fusion proteins from which the catalytic domains are derived. In some embodiments, the a CMP-sialic acid synthase polypeptide and a sialyltransferase polypeptide are fused to form a single polypeptide. Many sialyltransferase enzymes are known to those of skill and can be used in the methods of the invention. For example, a fusion between a Neisseria CMP-sialic acid synthase polypeptide and a Neisseria sialyltransferase protein is described in, e.g., WO99/31224 and Gilbert et al., Nat. Biotechnol. 16:769-72 (1998). Other fusions can be used in the invention, for example, between a Neisseria CMP-sialic acid synthase polypeptide and a Campylobacter sialyltransferase.

An “accessory enzyme,” as referred to herein, is an enzyme that is involved in catalyzing a reaction that, for example, forms a substrate or other reactant for a glycosyltransferase reaction. An accessory enzyme can, for example, catalyze the formation of a nucleotide sugar that is used as a sugar donor moiety by a glycosyltransferase. An accessory enzyme can also be one that is used in the generation of a nucleotide triphosphate that is required for formation of a nucleotide sugar, or in the generation of the sugar which is incorporated into the nucleotide sugar.

A “catalytic domain” refers to a portion of an enzyme that is sufficient to catalyze an enzymatic reaction that is normally carried out by the enzyme. For example, a catalytic domain of a sialyltransferase will include a sufficient portion of the sialyltransferase to transfer a sialic acid residue from a sugar donor to an acceptor saccharide. A catalytic domain can include an entire enzyme, a subsequence thereof, or can include additional amino acid sequences that are not attached to the enzyme or subsequence as found in nature.

The term “isolated” refers to material that is substantially or essentially free from components which interfere with the activity of an enzyme. For cells, saccharides, nucleic acids, and polypeptides of the invention, the term “isolated” refers to material that is substantially or essentially free from components which normally accompany the material as found in its native state. Typically, isolated saccharides, proteins or nucleic acids of the invention are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligonucleotides, or other sialylated products, purity can be determined using, e.g., thin layer chromatography, HPLC, or mass spectroscopy.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides, refers to two or more sequences or subsequences that have at least 60%, preferably 80% or 85%, most preferably at least 90%, 91%, 92%, 93%, 94%, 95%, 96%, 97%, 98%, or 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na⁺ ion, typically about 0.01 to 1.0 M Na⁺ ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90-95° C. for 30-120 sec, an annealing phase lasting 30-120 sec, and an extension phase of about 72° C. for 1-2 min. Protocols and guidelines for low and high stringency amplification reactions are available, e.g., in Innis, et al. (1990) PCR Protocols: A Guide to Methods and Applications Academic Press, N.Y.

The phrases “specifically binds to” or “specifically immunoreactive with”, when referring to an antibody refers to a binding reaction which is determinative of the presence of the protein or other antigen in the presence of a heterogeneous population of proteins, saccharides, and other biologics. Thus, under designated immunoassay conditions, the specified antibodies bind preferentially to a particular antigen and do not bind in a significant amount to other molecules present in the sample. Specific binding to an antigen under such conditions requires an antibody that is selected for its specificity for a particular antigen. A variety of immunoassay formats can be used to select antibodies specifically immunoreactive with a particular antigen. For example, solid-phase ELISA immunoassays are routinely used to select monoclonal antibodies specifically immunoreactive with an antigen. See Harlow and Lane (1988) Antibodies, A Laboratory Manual, Cold Spring Harbor Publications, New York, for a description of immunoassay formats and conditions that can be used to determine specific immunoreactivity.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F (ab)′₂, a dimer of Fab which itself is a light chain joined to V_{H}-C_{H}1 by a disulfide bond. The F (ab)′₂ may be reduced under mild conditions to break the disulfide linkage in the hinge region, thereby converting the F (ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

In one embodiment, the antibody is conjugated to an “effector” moiety. The effector moiety can be any number of molecules, including labeling moieties such as radioactive labels or fluorescent labels for use in diagnostic assays.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to IgE protein, polymorphic variants, alleles, orthologs, and conservatively modified variants, or splice variants, or portions thereof, can be selected to obtain only those polyclonal antibodies that are specifically immunoreactive with IgE proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

An “antigen” is a molecule that is recognized and bound by an antibody, e.g., peptides, carbohydrates, organic molecules, or more complex molecules such as glycolipids and glycoproteins. The part of the antigen that is the target of antibody binding is an antigenic determinant and a small functional group that corresponds to a single antigenic determinant is called a hapten.

A “label” is a composition detectable by spectroscopic, photochemical, biochemical, immunochemical, or chemical means. For example, useful labels include ³²P, ¹²⁵I, fluorescent dyes, electron-dense reagents, enzymes (e.g. as commonly used in an ELISA), biotin, digoxigenin, or haptens and proteins for which antisera or monoclonal antibodies are available (e.g., the polypeptide of SEQ ID NO:3 can be made detectable, e.g., by incorporating a radiolabel into the peptide, and used to detect antibodies specifically reactive with the peptide).

The term “immunoassay” is an assay that uses an antibody to specifically bind an antigen. The immunoassay is characterized by the use of specific binding properties of a particular antibody to isolate, target, and/or quantify the antigen.

The term “carrier molecule” means an immunogenic molecule containing antigenic determinants recognized by T cells. A carrier molecule can be a protein or can be a lipid. A carrier protein is conjugated to a polypeptide to render the polypeptide immunogenic. Carrier proteins include keyhole limpet hemocyanin, horseshoe crab hemocyanin, and bovine serum albumin.

The term “adjuvant” means a substance that nonspecifically enhances the immune response to an antigen. Adjuvants include Freund's adjuvant, either complete or incomplete; Titermax gold adjuvant; alum; and bacterial LPS.

### III. Sialyltransferase Polypeptides Comprising Conserved Sequence Motifs

The sialyltransferase polypeptides of the inventions comprise two motifs: sialyltransferase motif A, DVFRCNQFYFED/E, (SEQ ID NO: 1), and conservatively modified variants of that sequence and sialyltransferase motif B, RITSGVYMC, (SEQ ID NO:2), and conservatively modified variants of that sequence. In some embodiments, the sialyltransferase polypeptides comprise either the sialyltransferase motif A DVFRCNQFYFED or DVFRCNQFYFEE, and sialyltransferase motif B RITSGVYMC, (SEQ ID NO:2). The sialyltransferase polypeptides of the invention catalyze the transfer of a sialic acid moiety from a donor substrate to an acceptor substrate.

The conserved sialyltransferase motifs were identified by analysis of previously identified and newly discovered bacterial sialytransferases. The amino acid sequence of 18 sialyltransferases were aligned, and the conserved sialyltransferase sequence motifs A and B were identified by visual inspection. (See, e.g., FIG. 1.) FIG. 1 also provides a consensus sequence of the 18 sialyltransferase polypeptides. Those of skill will recognize that the position of amino acids in the consensus sequence can be used to identify an amino acid in a specific sialyltransferase polypeptide, even if the exact numbering of amino acid residues differs.

In some embodiments the sialyltransferase polypeptides also comprise other amino acid residues that appear to be important for enzymatic activity. For example, the structure of Cst-II from Campylobacter jejuni strain OH4384 has been solved. (See, e.g., Chiu et al., Nat. Struc. Mol. Biol. 11:163-170 (2004)). Mutational analysis of the Cst-II enzyme demonstrated that, for example the arginine residue of sialyltransferase motif B is required for activity. The arginine residue of sialyltransferase motif B is referred to as R129 in Cst-II and correlates to R165 of the sialyltransferase consensus sequence of FIG. 1. Other amino acid residues that appear to be important for catalytic activity include Cst-II Y156 (corresponding to consensus Y192), Cst-II Y162 (corresponding to consensus Y199) and Cst-II H188 (corresponding to consensus H226). Thus, in some embodiments, the sialyltransferase polypeptides comprise sialyltransferase motif A, sialyltransferase motif B and an amino acid residue corresponding to consensus Y192; or sialyltransferase motif A, sialyltransferase motif B and an amino acid residue corresponding to consensus Y192 and an amino acid residue corresponding to consensus Y199 or H226; or sialyltransferase motif A, sialyltransferase motif B and an amino acid residue corresponding to consensus Y199; or sialyltransferase motif A, sialyltransferase motif B and an amino acid residue corresponding to consensus Y199 and an amino acid residue corresponding to consensus H226; or sialyltransferase motif A, sialyltransferase motif B and an amino acid residue corresponding to consensus H226; sialyltransferase motif A, sialyltransferase motif B and an amino acid residue corresponding to consensus Y192, an amino acid residue corresponding to consensus Y199 and an amino acid residue corresponding to consensus H226.

Other amino acid residues can be important for enzymatic activity based on the structural data and can be included in sialyltransferase polypeptides with sialyltransferase motifs A and B, e.g., amino acid residues corresponding to consensus residues N44, N86, Q93, D190, F191, S198, F215, or Y222. Those of skill will note on reviewing FIG. 1, that at consensus residues S198, Y222, and F215, other amino acids can be tolerated. Also, N86 and Q93 are deleted from sialyltransferase polypeptides, e.g., from some H. influenzae sialyltransferase polypeptides. The above amino acids residues can be included in a sialyltransferase polypeptide, i.e., a polypeptide comprising sialyltransferase motifs A and B singly or in any combination, including combinations with amino acid residues corresponding to consensus Y192, Y199 or H226.

Examples of sialyltransferase polypeptides that comprise sialyltransferase motifs include e.g., Cst-I protein from C. jejuni strain O:19, Cst-I protein from C. jejuni strain O:36, Lic3A sialyltransferase protein from H. influenzae, and Lic3A2 sialyltransferase protein from H. influenzae.

### IV. Modifications of Sialyltransferase Polypeptides Comprising Conserved Sequence Motifs

The sialyltransferase polypeptides comprising conserved sequence motifs can also be modified, so long as they maintain sialyltransferase activity. Modifications include truncations, described supra, and, in some embodiments, site directed mutagenesis of the protein.

Site directed mutagenesis can be used to alter the acceptor specificity of a sialyltransferase polypeptide comprising conserved sequence motifs. Some sialytransferase polypeptides are able to sialylate an acceptor molecule by forming α2,3 and/or α2,8 linkages. For example CstII enzymes from C. jejuni strains OH4382, OH4384, O:10, and O:41 are all able to form α2,3 and/or α2,8 linkages. Mutation of Asn51 (corresponding to N86 of the consensus sequence) to a threonine residue eliminated the ability of CstII from OH4282, OH4384 to add sialic acid in an α2,8 linkage. However, mutation of Thr51 to asparagines in a monofunctional O:19 strain, resulted in an enzyme that was able to a sialic acid in both an α2,3 as well as an α2,8 linkage. (See, e.g., Gilbert et al., J. Biol. Chem. 277:327-337 (2002). Thus, mutation of the residue corresponding to position 86 of the consensus sequence can be used to alter the substrate specificity of a sialyltransferase polypeptide comprising conserved sequence motifs. In addition, a mutation of residue Ile53 (corresponding to residue 88 of the consensus sequence) to an glycine in CstII enzymes from C. jejuni strains OH4382, OH4384 resulted in large increases in enzymatic activity. Thus, mutation of the residue corresponding to position 88 of the consensus sequence can be used to alter the activity of a sialyltransferase polypeptide comprising conserved sequence motifs.

### V. Isolation of Nucleic Acids Encoding Sialyltransferase Polypeptides Comprising Conserved Sequence Motifs

Nucleic acids that encode sialyltransferase polypeptides comprising conserved sequence motifs include nucleic acids that encode the sialyltransferase polypeptides described above, i.e., sialyltransferase polypeptides that comprise sialyltransferase motif A, DVFRCNQFYFED/E, (SEQ ID NO: 1), and conservatively modified variants of that sequence and sialyltransferase motif B, RITSGVYMC, (SEQ ID NO:2), and conservatively modified variants of that sequence. In some embodiments, the sialyltransferase polypeptides comprise either the sialyltransferase motif A DVFRCNQFYFED or DVFRCNQFYFEE, and sialyltransferase motif B RITSGVYMC, (SEQ ID NO:2). The sialyltransferase polypeptides of the invention catalyze the transfer of a sialic acid moiety from a donor substrate to an acceptor substrate. The encoded sialyltransferase polypeptides can also comprise amino acid residues identified by structural analysis and that correspond to consensus amino acid residues Y192, Y199, H226, N44, N86, Q93, D190, F191, S198, F215, or Y222.

Examples of nucleic acids that encode sialyltransferase polypeptides comprising conserved sequence motifs include nucleic acids that encode Cst-I protein from C. jejuni strain O:19 and Cst-I protein from C. jejuni strain O:36.

Nucleic acids that encode sialyltransferase polypeptides comprising sialyltransferase motifs A and B, e.g., bacterial sialyltransferases, including sialyltransferases from Campylobacter, Haemophilus, and Pseudomonous species, and methods of obtaining such nucleic acids, are known to those of skill in the art. Suitable nucleic acids (e.g., cDNA, genomic, or subsequences (probes)) can be cloned, or amplified by in vitro methods such as the polymerase chain reaction (PCR), the ligase chain reaction (LCR), the transcription-based amplification system (TAS), or the self-sustained sequence replication system (SSR). A wide variety of cloning and in vitro amplification methodologies are well-known to persons of skill. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864.

A DNA that encodes a sialyltransferase polypeptide comprising sialyltransferase motifs A and B, or a subsequences thereof, can be prepared by any suitable method described above, including, for example, cloning and restriction of appropriate sequences with restriction enzymes. In one preferred embodiment, nucleic acids encoding sialyltransferase polypeptides comprising sialyltransferase motifs A and B are isolated by routine cloning methods. A nucleotide sequence of a sialyltransferase polypeptide comprising sialyltransferase motifs A and B as provided in, for example, FIG. 1 or other sequence database (see above) can be used to provide probes that specifically hybridize to a gene encoding a sialyltransferase polypeptide comprising sialyltransferase motifs A and B in a genomic DNA sample; or to an mRNA, encoding a sialyltransferase polypeptide comprising sialyltransferase motifs A and B, in a total RNA sample (e.g., in a Southern or Northern blot). Once the target nucleic acid encoding a sialyltransferase polypeptide comprising sialyltransferase motifs A and B is identified, it can be isolated according to standard methods known to those of skill in the art (see, e.g., Sambrook et al. (1989) Molecular Cloning: A Laboratory Manual, 2nd Ed., Vols. 1-3, Cold Spring Harbor Laboratory; Berger and Kimmel (1987) Methods in Enzymology, Vol. 152: Guide to Molecular Cloning Techniques, San Diego: Academic Press, Inc.; or Ausubel et al. (1987) Current Protocols in Molecular Biology, Greene Publishing and Wiley-Interscience, New York). Further, the isolated nucleic acids can be cleaved with restriction enzymes to create nucleic acids encoding the full-length sialyltransferase polypeptide comprising sialyltransferase motifs A and B, or subsequences thereof, e.g., containing subsequences encoding at least a subsequence of a catalytic domain of a sialyltransferase polypeptide comprising sialyltransferase motifs A and B. These restriction enzyme fragments, encoding a sialyltransferase polypeptide comprising sialyltransferase motifs A and B or subsequences thereof, may then be ligated, for example, to produce a nucleic acid encoding a sialyltransferase protein comprising sialyltransferase motifs A and B.

A nucleic acid encoding a sialyltransferase polypeptide comprising sialyltransferase motifs A and B, or a subsequence thereof, can be characterized by assaying for the expressed product. Assays based on the detection of the physical, chemical, or immunological properties of the expressed protein can be used. For example, one can identify a cloned sialyltransferase comprising sialyltransferase motifs A and B, by the ability of a protein encoded by the nucleic acid to catalyze the transfer of a sialic acid moiety from a donor substrate to an acceptor substrate. In one method, capillary electrophoresis is employed to detect the reaction products. This highly sensitive assay involves using either saccharide or disaccharide aminophenyl derivatives which are labeled with fluorescein as described in Wakarchuk et al. (1996) J. Biol. Chem. 271 (45): 28271-276. To assay for α2,3-sialyltransferase, Lac-FCHASE is used as a substrate. To assay for α2,8-sialyltransferase, GM3-FCHASE is used as a substrate. (See, e.g., U.S. Pat. No. 6,503,744, which is herein incorporated by reference.) The reaction products of other glycosyltransferases can be detected using capillary electrophoresis, e.g., to assay for a Neisseria lgtC enzyme, either FCHASE-AP-Lac or FCHASE-AP-Gal can be used, whereas for the Neisseria lgtB enzyme an appropriate reagent is FCHASE-AP-GlcNAc (Wakarchuk, supra). Other methods for detection of oligosaccharide reaction products include thin layer chromatography and GC/MS and are disclosed in U.S. Pat. No. 6,503,744, which is herein incorporated by reference.

Also, a nucleic acid encoding a sialyltransferase polypeptide comprising sialyltransferase motifs A and B, or a subsequence thereof, can be chemically synthesized. Suitable methods include the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill recognizes that while chemical synthesis of DNA is often limited to sequences of about 100 bases, longer sequences may be obtained by the ligation of shorter sequences.

Nucleic acids encoding sialyltransferase polypeptides comprising sialyltransferase motifs A and B, or subsequences thereof, can be cloned using DNA amplification methods such as polymerase chain reaction (PCR). Thus, for example, the nucleic acid sequence or subsequence is PCR amplified, using a sense primer containing one restriction enzyme site (e.g., NdeI) and an antisense primer containing another restriction enzyme site (e.g., HindIII). This will produce a nucleic acid encoding the desired sialyltransferase polypeptide comprising sialyltransferase motifs A and B or subsequence and having terminal restriction enzyme sites. This nucleic acid can then be easily ligated into a vector containing a nucleic acid encoding the second molecule and having the appropriate corresponding restriction enzyme sites. Suitable PCR primers can be determined by one of skill in the art using the sequence information provided in GenBank or other sources. Appropriate restriction enzyme sites can also be added to the nucleic acid encoding the sialyltransferase protein comprising sialyltransferase motifs A and B or a protein subsequence thereof by site-directed mutagenesis. The plasmid containing the sialyltransferase comprising sialyltransferase motifs A and B-encoding nucleotide sequence or subsequence is cleaved with the appropriate restriction endonuclease and then ligated into an appropriate vector for amplification and/or expression according to standard methods. Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

Some nucleic acids encoding bacterial sialyltransferase proteins comprising sialyltransferase motifs A and B can be amplified using PCR primers based on the sequence of previously identified sialyltransferase proteins, e.g., Cst-I, (see, e.g., U.S. Pat. No. 6,689,604); Cst-II, (see, e.g., U.S. Pat. No. 6,503,744); and Cst-III. Examples of PCR primers that can be used to amplify nucleic acid that encode sialyltransferase proteins comprising sialyltransferase motifs A and B include the following primer pairs:

### For Cst-I Nucleic Acids:

CJ18F:
5′ (41 mer, NdeI site in italics)
5′ C TTA GGA GGT CAT ATG ACA AGG ACT AGA ATG GAA
AAT GAA C 3′
and
CJ40R:
3′ with 6 His tail (60 mer, SalI site in italics.
(His)₆ tag in bold)
5′ CC TAG GTC GAC TCA TTA GTG GTG ATG GTG GTG ATG
TTC CCC TTT CTC AAA CTC TCT CTT C 3′;
For Cst-II nucleic acids:
CJ-131:
5′ CTTAGGAGGTCATATGAAAAAAGTTATTATTGCTGGAAATG 3′
and
CJ-132:
5′ CCTAGGTCGACTTATTTTCCTTTGAAATAATGCTTTATATC 3′;
For Cst-III nucleic acids:
CstH-5p:
5′ GGGGGGCATATGAGTATGAATATTAATGCTTTG 3′
and
CstH-3p:
5′ GGGGGGGTCGACTCATTATCTATTTTTATTTGCATATTTTTC 3′

In some bacteria, nucleic acids encoding sialyltransferase protein comprising sialyltransferase motifs A and B can be isolated by amplifying a specific chromosomal locus, e.g., the LOS locus of C. jejuni, and then identifying a sialyltransferase typically found at that locus (see, e.g., U.S. Pat. No. 6,503,744). Examples of PCR primers that can be used to amplify an LOS locus comprising nucleic acids encoding sialyltransferase protein comprising sialyltransferase motifs A and B include the following primer pairs:

CJ42: Primer in heptosylTase-II
5′ GC CAT TAC CGT ATC GCC TAA CCA GG 3′ 25 mer
CJ43: Primer in heptosylTase-I
5′ AAA GAA TAC GAA TTT GCT AAA GAG G 3′ 25 mer

Other physical properties of a recombinant sialyltransferase polypeptide comprising sialyltransferase motifs A and B expressed from a particular nucleic acid, can be compared to properties of known sialyltransferases to provide another method of identifying suitable sequences or domains of the sialyltransferase polypeptide comprising sialyltransferase motifs A and B that are determinants of acceptor substrate specificity and/or catalytic activity. Alternatively, a putative sialyltransferase polypeptide comprising sialyltransferase motifs A and B gene or recombinant sialyltransferase polypeptide comprising sialyltransferase motifs A and B gene can be mutated, and its role as a sialyltransferase, or the role of particular sequences or domains established by detecting a variation in the structure of a carbohydrate normally produced by the unmutated, naturally-occurring, or control sialyltransferase polypeptide. Those of skill will recognize that mutation or modification of sialyltransferase polypeptides of the invention can be facilitated by molecular biology techniques to manipulate the nucleic acids encoding the sialyltransferase polypeptides, e.g., PCR.

Functional domains of newly identified sialyltransferase polypeptides comprising sialyltransferase motifs A and B can be identified by using standard methods for mutating or modifying the polypeptides and testing them for activities such as acceptor substrate activity and/or catalytic activity, as described herein. The functional domains of the various sialyltransferase polypeptides comprising sialyltransferase motifs A and B can be used to construct nucleic acids encoding sialyltransferases comprising sialyltransferase motifs A and B and the functional domains of one or more sialyltransferase polypeptides. These multi-sialyltransferase fusion proteins can then be tested for the desired acceptor substrate or catalytic activity.

In an exemplary approach to cloning nucleic acids encoding sialyltransferase proteins comprising sialyltransferase motifs A and B, the known nucleic acid or amino acid sequences of cloned sialyltransferases are aligned and compared to determine the amount of sequence identity between various sialyltransferases. This information can be used to identify and select protein domains that confer or modulate sialyltransferase activities, e.g., acceptor substrate activity and/or catalytic activity based on the amount of sequence identity between the sialyltransferases of interest. For example, domains having sequence identity between the sialyltransferases of interest, and that are associated with a known activity, can be used to construct sialyltransferase proteins containing that domain and sialyltransferase motifs A and B, and having the activity associated with that domain (e.g., acceptor substrate specificity and/or catalytic activity).

### V. Expression of Sialyltransferase Polypeptides Comprising Conserved Sequence Motifs in Host Cells

Sialyltransferase proteins comprising sialyltransferase motifs A and B of the invention can be expressed in a variety of host cells, including E. coli, other bacterial hosts, and yeast. The host cells are preferably microorganisms, such as, for example, yeast cells, bacterial cells, or filamentous fungal cells. Examples of suitable host cells include, for example, Azotobacter sp. (e.g., A. vinelandii), Pseudomonas sp., Rhizobium sp., Erwinia sp., Escherichia sp. (e.g., E. coli), Bacillus, Pseudomonas, Proteus, Salmonella, Serratia, Shigella, Rhizobia, Vitreoscilla, Paracoccus and Klebsiella sp., among many others. The cells can be of any of several genera, including Saccharomyces (e.g., S. cerevisiae), Candida (e.g., C. utilis, C. parapsilosis, C. krusei, C. versatilis, C. lipolytica, C. zeylanoides, C. guilliermondii, C. albicans, and C. humicola), Pichia (e.g., P. farinosa and P. ohmeri), Torulopsis (e.g., T. candida, T. sphaerica, T. xylinus, T. famata, and T. versatilis), Debaryomyces (e.g., D. subglobosus, D. cantarellii, D. globosus, D. hansenii, and D. japonicus), Zygosaccharomyces (e.g., Z. rouxii and Z. bailii), Kluyveromyces (e.g., K. marxianus), Hansenula (e.g., H. anomala and H. jadinii), and Brettanomyces (e.g., B. lambicus and B. anomalus). Examples of useful bacteria include, but are not limited to, Escherichia, Enterobacter, Azotobacter, Erwinia, Klebsielia, Bacillus, Pseudomonas, Proteus, and Salmonella.

Once expressed in a host cell, the sialyltransferase polypeptides comprising sialyltransferase motifs A and B can be used to produced sialylated products. For example, the sialyltransferase polypeptides comprising sialyltransferase motifs A and B can be isolated using standard protein purification techniques and used in in vitro reactions described herein to make sialylated products. Partially purified sialyltransferase polypeptides comprising sialyltransferase motifs A and B can also be used in in vitro reactions to make sialylated products as can the permeabilized host cells. The host cells can also be used in an in vivo system (e.g., fermentative production) to produce sialylated products.

Typically, the polynucleotide that encodes the sialyltransferase polypeptides comprising sialyltransferase motifs A and B is placed under the control of a promoter that is functional in the desired host cell. An extremely wide variety of promoters are well known, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Accordingly, the invention provides expression cassettes into which the nucleic acids that encode fusion proteins are incorporated for high level expression in a desired host cell.

Expression control sequences that are suitable for use in a particular host cell are often obtained by cloning a gene that is expressed in that cell. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems (Change et al., Nature (1977) 198: 1056), the tryptophan (trp) promoter system (Goeddel et al., Nucleic Acids Res. (1980) δ: 4057), the tac promoter (DeBoer, et al., Proc. Natl. Acad. Sci. U.S.A. (1983) 80:21-25); and the lambda-derived P_{L} promoter and N-gene ribosome binding site (Shimatake et al., Nature (1981) 292: 128). The particular promoter system is not critical to the invention, any available promoter that functions in prokaryotes can be used.

For expression of sialyltransferase proteins comprising sialyltransferase motifs A and B in prokaryotic cells other than E. coli, a promoter that functions in the particular prokaryotic species is required. Such promoters can be obtained from genes that have been cloned from the species, or heterologous promoters can be used. For example, the hybrid trp-lac promoter functions in Bacillus in addition to E. coli.

A ribosome binding site (RBS) is conveniently included in the expression cassettes of the invention. An RBS in E. coli, for example, consists of a nucleotide sequence 3-9 nucleotides in length located 3-11 nucleotides upstream of the initiation codon (Shine and Dalgarno, Nature (1975) 254: 34; Steitz, In Biological regulation and development: Gene expression (ed. R. F. Goldberger), vol. 1, p. 349, 1979, Plenum Publishing, NY).

For expression of the sialyltransferase proteins comprising sialyltransferase motifs A and B in yeast, convenient promoters include GAL1-10 (Johnson and Davies (1984) Mol.

Cell. Biol. 4:1440-1448) ADH2 (Russell et al. (1983) J. Biol. Chem. 258:2674-2682), PHO5 (EMBO J. (1982) 6:675-680), and MFα (Herskowitz and Oshima (1982) in The Molecular Biology of the Yeast Saccharomyces (eds. Strathem, Jones, and Broach) Cold Spring Harbor Lab., Cold Spring Harbor, N.Y., pp. 181-209). Another suitable promoter for use in yeast is the ADH2/GAPDH hybrid promoter as described in Cousens et al., Gene 61:265-275 (1987). For filamentous fungi such as, for example, strains of the fungi Aspergillus (McKnight et al., U.S. Pat. No. 4,935,349), examples of useful promoters include those derived from Aspergillus nidulans glycolytic genes, such as the ADH3 promoter (McKnight et al., EMBO J. 4: 2093 2099 (1985)) and the tpiA promoter. An example of a suitable terminator is the ADH3 terminator (McKnight et al.).

Either constitutive or regulated promoters can be used in the present invention. Regulated promoters can be advantageous because the host cells can be grown to high densities before expression of the fusion proteins is induced. High level expression of heterologous proteins slows cell growth in some situations. An inducible promoter is a promoter that directs expression of a gene where the level of expression is alterable by environmental or developmental factors such as, for example, temperature, pH, anaerobic or aerobic conditions, light, transcription factors and chemicals. Such promoters are referred to herein as “inducible” promoters, which allow one to control the timing of expression of the glycosyltransferase or enzyme involved in nucleotide sugar synthesis. For E. coli and other bacterial host cells, inducible promoters are known to those of skill in the art. These include, for example, the lac promoter, the bacteriophage lambda P_{L} promoter, the hybrid trp-lac promoter (Amann et al. (1983) Gene 25: 167; de Boer et al. (1983) Proc. Nat'l. Acad. Sci. USA 80: 21), and the bacteriophage T7 promoter (Studier et al. (1986) J. Mol. Biol.; Tabor et al. (1985) Proc. Nat'l Acad. Sci. USA 82: 1074-8). These promoters and their use are discussed in Sambrook et al., supra. A particularly preferred inducible promoter for expression in prokaryotes is a dual promoter that includes a tac promoter component linked to a promoter component obtained from a gene or genes that encode enzymes involved in galactose metabolism (e.g., a promoter from a UDP galactose 4-epimerase gene (galE)). The dual tac-gal promoter, which is described in PCT Patent Application Publ. No. WO98/20111,

A construct that includes a polynucleotide of interest operably linked to gene expression control signals that, when placed in an appropriate host cell, drive expression of the polynucleotide is termed an “expression cassette.” Expression cassettes that encode the fusion proteins of the invention are often placed in expression vectors for introduction into the host cell. The vectors typically include, in addition to an expression cassette, a nucleic acid sequence that enables the vector to replicate independently in one or more selected host cells. Generally, this sequence is one that enables the vector to replicate independently of the host chromosomal DNA, and includes origins of replication or autonomously replicating sequences. Such sequences are well known for a variety of bacteria. For instance, the origin of replication from the plasmid pBR322 is suitable for most Gram-negative bacteria. Alternatively, the vector can replicate by becoming integrated into the host cell genomic complement and being replicated as the cell undergoes DNA replication. A preferred expression vector for expression of the enzymes is in bacterial cells is pTGK, which includes a dual tac-gal promoter and is described in PCT Patent Application Publ. NO. WO98/20111.

The construction of polynucleotide constructs generally requires the use of vectors able to replicate in bacteria. A plethora of kits are commercially available for the purification of plasmids from bacteria (see, for example, EasyPrepJ, FlexiPrepJ, both from Pharmacia Biotech; StrataCleanJ, from Stratagene; and, QIAexpress Expression System, Qiagen). The isolated and purified plasmids can then be further manipulated to produce other plasmids, and used to transfect cells. Cloning in Streptomyces or Bacillus is also possible.

Selectable markers are often incorporated into the expression vectors used to express the polynucleotides of the invention. These genes can encode a gene product, such as a protein, necessary for the survival or growth of transformed host cells grown in a selective culture medium. Host cells not transformed with the vector containing the selection gene will not survive in the culture medium. Typical selection genes encode proteins that confer resistance to antibiotics or other toxins, such as ampicillin, neomycin, kanamycin, chloramphenicol, or tetracycline. Alternatively, selectable markers may encode proteins that complement auxotrophic deficiencies or supply critical nutrients not available from complex media, e.g., the gene encoding D-alanine racemase for Bacilli. Often, the vector will have one selectable marker that is functional in, e.g., E. coli, or other cells in which the vector is replicated prior to being introduced into the host cell. A number of selectable markers are known to those of skill in the art and are described for instance in Sambrook et al., supra.

Construction of suitable vectors containing one or more of the above listed components employs standard ligation techniques as described in the references cited above. Isolated plasmids or DNA fragments are cleaved, tailored, and re-ligated in the form desired to generate the plasmids required. To confirm correct sequences in plasmids constructed, the plasmids can be analyzed by standard techniques such as by restriction endonuclease digestion, and/or sequencing according to known methods. Molecular cloning techniques to achieve these ends are known in the art. A wide variety of cloning and in vitro amplification methods suitable for the construction of recombinant nucleic acids are well-known to persons of skill. Examples of these techniques and instructions sufficient to direct persons of skill through many cloning exercises are found in Berger and Kimmel, Guide to Molecular Cloning Techniques, Methods in Enzymology, Volume 152, Academic Press, Inc., San Diego, Calif. (Berger); and Current Protocols in Molecular Biology, F. M. Ausubel et al., eds., Current Protocols, a joint venture between Greene Publishing Associates, Inc. and John Wiley & Sons, Inc., (1998 Supplement) (Ausubel).

A variety of common vectors suitable for use as starting materials for constructing the expression vectors of the invention are well known in the art. For cloning in bacteria, common vectors include pBR322 derived vectors such as pBLUESCRIPT™, and λ-phage derived vectors. In yeast, vectors include Yeast Integrating plasmids (e.g., YIp5) and Yeast Replicating plasmids (the YRp series plasmids) and pGPD-2. Expression in mammalian cells can be achieved using a variety of commonly available plasmids, including pSV2, pBC12BI, and p91023, as well as lytic virus vectors (e.g., vaccinia virus, adeno virus, and baculovirus), episomal virus vectors (e.g., bovine papillomavirus), and retroviral vectors (e.g., murine retroviruses).

The methods for introducing the expression vectors into a chosen host cell are not particularly critical, and such methods are known to those of skill in the art. For example, the expression vectors can be introduced into prokaryotic cells, including E. coli, by calcium chloride transformation, and into eukaryotic cells by calcium phosphate treatment or electroporation. Other transformation methods are also suitable.

Translational coupling may be used to enhance expression. The strategy uses a short upstream open reading frame derived from a highly expressed gene native to the translational system, which is placed downstream of the promoter, and a ribosome binding site followed after a few amino acid codons by a termination codon. Just prior to the termination codon is a second ribosome binding site, and following the termination codon is a start codon for the initiation of translation. The system dissolves secondary structure in the RNA, allowing for the efficient initiation of translation. See Squires, et. al. (1988), J. Biol. Chem. 263: 16297-16302.

The sialyltransferase polypeptides comprising sialyltransferase motifs A and B can be expressed intracellularly, or can be secreted from the cell. Intracellular expression often results in high yields. If necessary, the amount of soluble, active fusion protein may be increased by performing refolding procedures (see, e.g., Sambrook et al., supra.; Marston et al., Bio/Technology (1984) 2: 800; Schoner et al., Bio/Technology (1985) 3: 151). In embodiments in which the sialyltransferase polypeptides comprising sialyltransferase motifs A and B are secreted from the cell, either into the periplasm or into the extracellular medium, the DNA sequence is linked to a cleavable signal peptide sequence. The signal sequence directs translocation of the fusion protein through the cell membrane. An example of a suitable vector for use in E. coli that contains a promoter-signal sequence unit is pTA1529, which has the E. coli phoA promoter and signal sequence (see, e.g., Sambrook et al., supra.; Oka et al., Proc. Natl. Acad. Sci. USA (1985) 82: 7212; Talmadge et al., Proc. Natl. Acad. Sci. USA (1980) 77: 3988; Takahara et al., J. Biol. Chem. (1985) 260: 2670). In another embodiment, the fusion proteins are fused to a subsequence of protein A or bovine serum albumin (BSA), for example, to facilitate purification, secretion, or stability.

The sialyltransferase polypeptides comprising sialyltransferase motifs A and B of the invention can also be further linked to other bacterial proteins. This approach often results in high yields, because normal prokaryotic control sequences direct transcription and translation. In E. coli, lacZ fusions are often used to express heterologous proteins. Suitable vectors are readily available, such as the pUR, pEX, and pMR100 series (see, e.g., Sambrook et al., supra.). For certain applications, it may be desirable to cleave the non-glycosyltransferase and/or accessory enzyme amino acids from the fusion protein after purification. This can be accomplished by any of several methods known in the art, including cleavage by cyanogen bromide, a protease, or by Factor Xₐ (see, e.g., Sambrook et al., supra.; Itakura et al., Science (1977) 198: 1056; Goeddel et al., Proc. Natl. Acad. Sci. USA (1979) 76: 106; Nagai et al., Nature (1984) 309: 810; Sung et al., Proc. Natl. Acad. Sci. USA (1986) 83: 561). Cleavage sites can be engineered into the gene for the fusion protein at the desired point of cleavage.

More than one recombinant protein may be expressed in a single host cell by placing multiple transcriptional cassettes in a single expression vector, or by utilizing different selectable markers for each of the expression vectors which are employed in the cloning strategy.

A suitable system for obtaining recombinant proteins from E. coli which maintains the integrity of their N-termini has been described by Miller et al. Biotechnology 7:698-704 (1989). In this system, the gene of interest is produced as a C-terminal fusion to the first 76 residues of the yeast ubiquitin gene containing a peptidase cleavage site. Cleavage at the junction of the two moieties results in production of a protein having an intact authentic N-terminal reside.

### VI. Purification of Sialyltransferase Polypeptides Comprising Conserved Sequence Motifs

The sialyltransferase proteins of the present invention can be expressed as intracellular proteins or as proteins that are secreted from the cell, and can be used in this form, in the methods of the present invention. For example, a crude cellular extract containing the expressed intracellular or secreted sialyltransferase polypeptide comprising sialyltransferase motifs A and B can used in the methods of the present invention.

Alternatively, the sialyltransferase polypeptide comprising sialyltransferase motifs A and B can be purified according to standard procedures of the art, including ammonium sulfate precipitation, affinity columns, column chromatography, gel electrophoresis and the like (see, generally, R. Scopes, Protein Purification, Springer-Verlag, N.Y. (1982), Deutscher, Methods in Enzymology Vol. 182: Guide to Protein Purification., Academic Press, Inc. N.Y. (1990)). Substantially pure compositions of at least about 70, 75, 80, 85, 90% homogeneity are preferred, and 92, 95, 98 to 99% or more homogeneity are most preferred. The purified proteins may also be used, e.g., as immunogens for antibody production.

To facilitate purification of the sialyltransferases polypeptides comprising sialyltransferase motifs A and B of the invention, the nucleic acids that encode the proteins can also include a coding sequence for an epitope or “tag” for which an affinity binding reagent is available, i.e. a purification tag. Examples of suitable epitopes include the myc and V-5 reporter genes; expression vectors useful for recombinant production of fusion proteins having these epitopes are commercially available (e.g., Invitrogen (Carlsbad Calif.) vectors pcDNA3.1/Myc-His and pcDNA3.11V5-His are suitable for expression in mammalian cells). Additional expression vectors suitable for attaching a tag to the sialyltransferases polypeptide comprising sialyltransferase motifs A and B proteins of the invention, and corresponding detection systems are known to those of skill in the art, and several are commercially available (e.g., FLAG” (Kodak, Rochester N.Y.). Another example of a suitable tag is a polyhistidine sequence, which is capable of binding to metal chelate affinity ligands. Typically, six adjacent histidines are used, although one can use more or less than six. Suitable metal chelate affinity ligands that can serve as the binding moiety for a polyhistidine tag include nitrilo-tri-acetic acid (NTA) (Hochuli, E. (1990) “Purification of recombinant proteins with metal chelating adsorbents” In Genetic Engineering: Principles and Methods, J. K. Setlow, Ed., Plenum Press, NY; commercially available from Qiagen (Santa Clarita, Calif.)).

Purification tags also include maltose binding domains and starch binding domains. Purification of maltose binding domain proteins is know to those of skill in the art. Starch binding domains are described in WO 99/15636, herein incorporated by reference. Affinity purification of a fusion protein comprising a starch binding domain using a betacylodextrin (BCD)-derivatized resin is described in U.S. Ser. No. 60/468,374, filed May 5, 2003, herein incorporated by reference in its entirety.

Other haptens that are suitable for use as tags are known to those of skill in the art and are described, for example, in the Handbook of Fluorescent Probes and Research Chemicals (6th Ed., Molecular Probes, Inc., Eugene Oreg.). For example, dinitrophenol (DNP), digoxigenin, barbiturates (see, e.g., U.S. Pat. No. 5,414,085), and several types of fluorophores are useful as haptens, as are derivatives of these compounds. Kits are commercially available for linking haptens and other moieties to proteins and other molecules. For example, where the hapten includes a thiol, a heterobifunctional linker such as SMCC can be used to attach the tag to lysine residues present on the capture reagent.

One of skill would recognize that modifications can be made to the catalytic or functional domains of the sialyltransferase polypeptide comprising sialyltransferase motifs A and B without diminishing their biological activity. Some modifications may be made to facilitate the cloning, expression, or incorporation of the catalytic domain into a fusion protein. Such modifications are well known to those of skill in the art and include, for example, the addition of codons at either terminus of the polynucleotide that encodes the catalytic domain to provide, for example, a methionine added at the amino terminus to provide an initiation site, or additional amino acids (e.g., poly His) placed on either terminus to create conveniently located restriction enzyme sites or termination codons or purification sequences.

### VII. Fusion Sialyltransferase Proteins

In some embodiments, the recombinant cells of the invention express fusion proteins that have more than one enzymatic activity that is involved in synthesis of a desired sialylated oligosaccharide. The fusion polypeptides can be composed of, for example, a sialyltransferase polypeptide comprising sialyltransferase motifs A and B that is joined to a an accessory enzyme, e.g., CMP-sialic acid synthase. Fusion proteins can also be made using catalytic domains or other truncations of the enzymes. For example, a polynucleotide that encodes a sialyltransferase polypeptide comprising sialyltransferase motifs A and B can be joined, in-frame, to a polynucleotide that encodes an enzyme involved in CMP-sialic acid synthesis. The resulting fusion protein can then catalyze not only the synthesis of the activated sialic acid molecule, but also the transfer of the sialic acid moiety to the acceptor molecule. The fusion protein can be two or more sialic acid cycle enzymes linked into one expressible nucleotide sequence. The fusion sialyltransferase polypeptides of the present invention can be readily designed and manufactured utilizing various recombinant DNA techniques well known to those skilled in the art. Exemplary fusion proteins are described in PCT Patent Application PCT/CA98/01180, which was published as WO99/31224 on Jun. 24, 1999 and which discloses CMP-sialic acid synthase from Neisseria fused with an α2,3-sialyltransferase from Neisseria. Those of skill will recognize that many other CMP-sialic acid synthase polypeptides and sialyltransferases can be fused for use in the invention. In some embodiments, a CMP-sialic acid synthase from Neisseria is fused to a sialyltransferase from C. jejuni. The C. jejuni sialyltransferase (Cst) can be a CstI, CstII, or CstIII enzyme. A full-length or truncated version of the C. jejuni sialyltransferase polypeptide can be used in the fusion sialyltransferase protein. In some embodiments, more that one fusion sialyltransferase polypeptide is expressed in the cell.

In some embodiments, the recombinant cells of the invention express fusion proteins that have more than one enzymatic activity that is involved in addition of at least one additional sugar residue, e.g., a non-sialic acid residue. These fusion polypeptides can be composed of, for example, a catalytic domain of a glycosyltransferase, e.g., not a sialyltransferase, that is joined to a catalytic domain of an accessory enzyme. The accessory enzyme catalytic domain can, for example, catalyze a step in the formation of a nucleotide sugar which is a donor for the glycosyltransferase, or catalyze a reaction involved in a glycosyltransferase cycle. For example, a polynucleotide that encodes a glycosyltransferase can be joined, in-frame, to a polynucleotide that encodes an enzyme involved in nucleotide sugar synthesis. The resulting fusion protein can then catalyze not only the synthesis of the nucleotide sugar, but also the transfer of the sugar moiety to the acceptor molecule. The fusion protein can be two or more cycle enzymes linked into one expressible nucleotide sequence. The polypeptides of the present invention can be readily designed and manufactured utilizing various recombinant DNA techniques well known to those skilled in the art. Suitable fusion proteins are described in PCT Patent Application PCT/CA98/01180, which was published as WO99/31224 on Jun. 24, 1999, and include e.g., a UDP glucose epimerase fused in frame to a galactosyltransferase.

### VIII. Donor Substrates and Acceptor Substrates

Suitable donor substrates used by the sialyltransferase polypeptides comprising sialyltransferase motifs A and B and other glycosyltransferases in the methods of the invention include, but are not limited to, UDP-Glc, UDP-GlcNAc, UDP-Gal, UDP-GalNAc, GDP-Man, GDP-Fuc, UDP-GlcUA, and CMP-sialic acid and other activated sialic acid moieties. Guo et al., Applied Biochem. and Biotech. 68: 1-20 (1997)

Typically, acceptor substrates include a terminal galactose residue for addition of a sialic acid residue by an α2,3 linkage. For addition of a sialic acid residue in an α2,8 linkage, a second sialic acid residue is linked to a first sialic acid by an α2,8 linkage. Examples of suitable acceptors include a terminal Gal that is linked to GlcNAc or Glc by a β1,4 linkage, and a terminal Gal that is β1,3-linked to either GlcNAc or GalNAc. Suitable acceptors, include, for example, galactosyl acceptors such as Galβ1,4GlcNAc, Galβ1,4GalNAc, Galβ1,3GalNAc, lacto-N-tetraose, Galβ1,3GlcNAc, Galβ1,3Ara, Galβ1,6GlcNAc, Galβ1,4Glc (lactose), and other acceptors known to those of skill in the art (see, e.g., Paulson et al., J. Biol. Chem. 253: 5617-5624 (1978)). The terminal residue to which the sialic acid is attached can itself be attached to, for example, H, a saccharide, oligosaccharide, or an aglycone group having at least one carbohydrate atom. In some embodiments, the acceptor residue is a portion of an oligosaccharide that is attached to a protein, lipid, or proteoglycan, for example.

Suitable acceptor substrates used by the sialyltransferase polypeptides comprising sialyltransferase motifs A and B and methods of the invention include, but are not limited to, polysaccharides and oligosaccharides. For example, lactose can be sialylated to form a sialylactose, e.g. 3′ sialylactose. The sialyltransferases described herein can also be used in multienzyme systems to produce a desired product from a convenient starting material.

Suitable acceptor substrates used by the sialyltransferase polypeptides comprising sialyltransferase motifs A and B and methods of the invention include, but are not limited to, proteins, lipids, gangliosides and other biological structures (e.g., whole cells) that can be modified by the methods of the invention. These acceptor substrates will typically comprise the polysaccharide or oligosaccharide molecules described above. Exemplary structures, which can be modified by the methods of the invention include any a of a number glycolipids, glycoproteins and carbohydrate structures on cells known to those skilled in the art as set forth is Table 1.

TABLE 1

Hormones and Growth Factors
G-CSF
GM-CSF
TPO
EPO
EPO variants
α-TNF
Leptin
Enzymes and Inhibitors
t-PA
t-PA variants
Urokinase
Factors VII, VIII, IX, X
DNase
Glucocerebrosidase
Hirudin
α1 antitrypsin
Antithrombin III
Cytokines and Chimeric Cytokines
Interleukin-1 (IL-1), 1B, 2, 3, 4
Interferon-α (IFN-α)
IFN-α-2b
IFN-β
IFN-γ
Chimeric diptheria toxin-IL-2
Receptors and Chimeric Receptors
CD4
Tumor Necrosis Factor (TNF) receptor
Alpha-CD20
MAb-CD20
MAb-alpha-CD3
MAb-TNF receptor
MAb-CD4
PSGL-1
MAb-PSGL-1
Complement
GlyCAM or its chimera
N-CAM or its chimera
LFA-3
CTLA-IV
Monoclonal Antibodies (Immunoglobulins)
MAb-anti-RSV
MAb-anti-IL-2 receptor
MAb-anti-CEA
MAb-anti-platelet IIb/IIIa receptor
MAb-anti-EGF
MAb-anti-Her-2 receptor
Cells
Red blood cells
White blood cells (e.g., T cells, B cells,
dendritic cells, macrophages, NK cells,
neutrophils, monocytes and the like
Stem cells

Examples of suitable acceptor substrates used in sialyltransferase-catalyzed reactions, and examples of suitable acceptor substrates used in sialyltransferase-catalyzed reactions are described in Guo et al., Applied Biochem. and Biotech. 68: 1-20 (1997), but are not limited thereto.

The present invention provides sialyltransferase polypeptides comprising sialyltransferase motifs A and B that are selected for their ability to produce oligosaccharides, glycoproteins and glycolipids having desired oligosaccharide moieties. Similarly, if present, accessory enzymes are chosen based on an desired activated sugar substrate or on a sugar found on the product oligosaccharide.

For synthesis of glycoproteins, one can readily identify suitable sialyltransferase polypeptides comprising sialyltransferase motifs A and B by reacting various amounts of a sialyltransferase polypeptide comprising sialyltransferase motifs A and B of interest (e.g., 0.01-100 mU/mg protein) with a glycoprotein (e.g., at 1-10 mg/ml) to which is linked an oligosaccharide that has a potential acceptor site for glycosylation by the sialyltransferase of interest. The abilities of the recombinant sialyltransferases proteins of the present invention to add a sugar residue at the desired acceptor site are compared, and a sialyltransferase polypeptide comprising sialyltransferase motifs A and B having the desired property (e.g., acceptor substrate specificity or catalytic activity) is selected.

In general, the efficacy of the enzymatic synthesis of oligosaccharides, glycoproteins, and glycolipids, having desired sialylated oligosaccharide moieties, can be enhanced through use of recombinantly produced sialyltransferase polypeptides comprising sialyltransferase motifs A and B of the present invention. Recombinant techniques enable production of the recombinant sialyltransferase polypeptides comprising sialyltransferase motifs A and B in the large amounts that are required for large-scale in vitro glycoprotein and glycolipid modification.

In some embodiments, suitable oligosaccharides, glycoproteins, and glycolipids for use by the sialyltransferase polypeptides comprising sialyltransferase motifs A and B and methods of the invention can be glycoproteins and glycolipids immobilized on a solid support during the glycosylation reaction. The term “solid support” also encompasses semi-solid supports. Preferably, the target glycoprotein or glycolipid is reversibly immobilized so that the respective glycoprotein or glycolipid can be released after the glycosylation reaction is completed. Many suitable matrices are known to those of skill in the art. Ion exchange, for example, can be employed to temporarily immobilize a glycoprotein or glycolipid on an appropriate resin while the glycosylation reaction proceeds. A ligand that specifically binds to the glycoprotein or glycolipid of interest can also be used for affinity-based immobilization. For example, antibodies that specifically bind to a glycoprotein are suitable. Also, where the glycoprotein of interest is itself an antibody or contains a fragment thereof, one can use protein A or G as the affinity resin. Dyes and other molecules that specifically bind to a glycoprotein or glycolipid of interest are also suitable.

Preferably, when the acceptor saccharide is a truncated version of the full-length glycoprotein, it preferably includes the biologically active subsequence of the full-length glycoprotein. Exemplary biologically active subsequences include, but are not limited to, enzyme active sites, receptor binding sites, ligand binding sites, complementarity determining regions of antibodies, and antigenic regions of antigens.

### IX. Production of Sialylated Products

Sialyltransferase polypeptides comprising sialyltransferase motifs A and B can be used to make sialylated products in in vitro reactions mixes or by in vivo reactions, e.g., by fermentative growth of recombinant microorganisms that comprise nucleotides that encode sialyltransferase polypeptides comprising sialyltransferase motifs A and B.

A. In Vitro Reactions

The sialyltransferase polypeptides comprising sialyltransferase motifs A and B can be used to make sialylated products in in vitro reactions mixes. The in vitro reaction mixtures can include permeabilized microorganisms comprising the sialyltransferase polypeptides, partially purified sialytransferase polypeptides, or purified sialyltransferase polypeptides; as well as donor substrates acceptor substrates, and appropriate reaction buffers. For in vitro reactions, the recombinant glycosyltransferase proteins, such as sialyltransferase polypeptides comprising sialyltransferase motifs A and B, acceptor substrates, donor substrates and other reaction mixture ingredients are combined by admixture in an aqueous reaction medium. Additional glycosyltransferases can be used in combination with the sialyltransferase polypeptides comprising sialyltransferase motifs A and B, depending on the desired sialylated product. The medium generally has a pH value of about 5 to about 8.5. The selection of a medium is based on the ability of the medium to maintain pH value at the desired level. Thus, in some embodiments, the medium is buffered to a pH value of about 7.5. If a buffer is not used, the pH of the medium should be maintained at about 5 to 8.5, depending upon the particular glycosyltransferase used. For fucosyltransferases, the pH range is preferably maintained from about 6.0 to 8.0. For sialyltransferases, the range is preferably from about 5.5 to about 8.0.

Enzyme amounts or concentrations are expressed in activity units, which is a measure of the initial rate of catalysis. One activity unit catalyzes the formation of 1 μmol of product per minute at a given temperature (typically 37° C.) and pH value (typically 7.5). Thus, 10 units of an enzyme is a catalytic amount of that enzyme where 10 μmol of substrate are converted to 10 μmol of product in one minute at a temperature of 37° C. and a pH value of 7.5.

The reaction mixture may include divalent metal cations (Mg²⁺, Mn²⁺). The reaction medium may also comprise solubilizing detergents (e.g., Triton or SDS) and organic solvents such as methanol or ethanol, if necessary. The enzymes can be utilized free in solution or can be bound to a support such as a polymer. The reaction mixture is thus substantially homogeneous at the beginning, although some precipitate can form during the reaction.

The temperature at which an above process is carried out can range from just above freezing to the temperature at which the most sensitive enzyme denatures. That temperature range is preferably about 0° C. to about 45° C., and more preferably at about 20° C. to about 37° C.

The reaction mixture so formed is maintained for a period of time sufficient to obtain the desired high yield of desired oligosaccharide determinants present on oligosaccharide groups attached to the glycoprotein to be glycosylated. For large-scale preparations, the reaction will often be allowed to proceed for between about 0.5-240 hours, and more typically between about 1-18 hours.

One or more of the glycosyltransferase reactions can be carried out as part of a glycosyltransferase cycle. Preferred conditions and descriptions of glycosyltransferase cycles have been described. A number of glycosyltransferase cycles (for example, sialyltransferase cycles, galactosyltransferase cycles, and fucosyltransferase cycles) are described in U.S. Pat. No. 5,374,541 and WO 9425615 A. Other glycosyltransferase cycles are described in Ichikawa et al. J. Am. Chem. Soc. 114:9283 (1992), Wong et al. J. Org. Chem. 57: 4343 (1992), DeLuca, et al., J. Am. Chem. Soc. 117:5869-5870 (1995), and Ichikawa et al. In Carbohydrates and Carbohydrate Polymers. Yaltami, ed. (ATL Press, 1993).

Other glycosyltransferases can be substituted into similar transferase cycles as have been described in detail for the fucosyltransferases and sialyltransferases. In particular, the glycosyltransferase can also be, for instance, glucosyltransferases, e.g., Alg8 (Stagljov et al., Proc. Natl. Acad. Sci. USA 91:5977 (1994)) or Alg5 (Heesen et al. Eur. J. Biochem. 224:71 (1994)), N-acetylgalactosaminyltransferases such as, for example, α(1,3) N-acetylgalactosaminyltransferase, β(1,4) N-acetylgalactosaminyltransferases (Nagata et al. J. Biol. Chem. 267:12082-12089 (1992) and Smith et al. J Biol. Chem. 269:15162 (1994)) and polypeptide N-acetylgalactosaminyltransferase (Homa et al. J Biol. Chem. 268:12609 (1993)). Suitable N-acetylglucosaminyltransferases include GnTI (2.4.1.101, Hull et al., BBRC 176:608 (1991)), GnTII, and GnTIII (Ihara et al. J. Biochem. 113:692 (1993)), GnTV (Shoreiban et al. J. Biol. Chem. 268: 15381 (1993)), O-linked N-acetylglucosaminyltransferase (Bierhuizen et al. Proc. Natl. Acad. Sci. USA 89:9326 (1992)), N-acetylglucosamine-1-phosphate transferase (Rajput et al. Biochem J. 285:985 (1992), and hyaluronan synthase. Suitable mannosyltransferases include α(1,2) mannosyltransferase, α(1,3) mannosyltransferase, β(1,4) mannosyltransferase, Dol-P-Man synthase, OCh1, and Pmt1.

For the above glycosyltransferase cycles, the concentrations or amounts of the various reactants used in the processes depend upon numerous factors including reaction conditions such as temperature and pH value, and the choice and amount of acceptor saccharides to be glycosylated. Because the glycosylation process permits regeneration of activating nucleotides, activated donor sugars and scavenging of produced PPi in the presence of catalytic amounts of the enzymes, the process is limited by the concentrations or amounts of the stoichiometric substrates discussed before. The upper limit for the concentrations of reactants that can be used in accordance with the method of the present invention is determined by the solubility of such reactants.

Preferably, the concentrations of activating nucleotides, phosphate donor, the donor sugar and enzymes are selected such that glycosylation proceeds until the acceptor is consumed. The considerations discussed below, while in the context of a sialyltransferase, are generally applicable to other glycosyltransferase cycles.

Each of the enzymes is present in a catalytic amount. The catalytic amount of a particular enzyme varies according to the concentration of that enzyme's substrate as well as to reaction conditions such as temperature, time and pH value. Means for determining the catalytic amount for a given enzyme under preselected substrate concentrations and reaction conditions are well known to those of skill in the art.

B. In Vivo Reactions

The sialyltransferase polypeptides comprising sialyltransferase motifs A and B can be used to make sialylated products by in vivo reactions, e.g., fermentative growth of recombinant microorganisms comprising the sialyltransferase polypeptides. Fermentative growth of recombinant microorganisms can occur in the presence of medium that includes an acceptor substrate and a donor substrate or a precursor to a donor substrate, e.g., sialic acid. See, e.g., Priem et al., Glycobiology 12:235-240 (2002). The microorganism takes up the acceptor substrate and the donor substrate or the precursor to a donor substrate and the addition of the donor substrate to the acceptor substrate takes place in the living cell. The microorganism can be altered to facilitate uptake of the acceptor substrate, e.g., by expressing a sugar transport protein. For example, where lactose is the acceptor saccharide, E. coli cells that express the LacY permease can be used. Other methods can be used to decrease breakdown of an acceptor saccharide or to increase production of a donor saccharide or a precursor of the donor saccharide. In some embodiments, production of sialylated products is enhanced by manipulation of the host microorganism. For example, in E. coli, break down of sialic acid can be minimized by using a host strain that is lack CMP-sialate synthase (NanA-). (In E. coli, CMP-sialate synthase appears to be a catabolic enzyme.) Also in E. coli, when lactose is, for example, the acceptor saccharide or an intermediate in synthesizing the sialylated product, lactose breakdown can be minimized by using host cells that are LacZ-.

C. Characterization of and Isolation of Sialylated Products

The production of sialylated products can be monitored by e.g., determining that production of the desired product has occurred or by determining that a substrate such as the acceptor substrate has been depleted. Those of skill will recognize that sialylated products such as oligosaccharide, can be identified using techniques such as chromatography, e.g., using paper or TLC plates, or by mass spectrometry, e.g., MALDI-TOF spectrometry, or by NMR spectroscopy. Methods of identification of sialylated products are known to those of skill in the art and are found, e.g., in U.S. Pat. No. 6,699,705, which is herein incorporated by reference for all purposes and in Varki et al., Preparation and Analysis of Glycoconjugates, in Current Protocols in Molecular Biology, Chapter 17 (Ausubel et al. eds, 1993).

In some embodiments, the sialyltransferase polypeptides comprising sialyltransferase motifs A and B and methods of the present invention are used to enzymatically synthesize a glycoprotein or glycolipid that has a substantially uniform glycosylation pattern. The glycoproteins and glycolipids include a saccharide or oligosaccharide that is attached to a protein, glycoprotein, lipid, or glycolipid for which a glycoform alteration is desired. The saccharide or oligosaccharide includes a structure that can function as an acceptor substrate for a glycosyltransferase. When the acceptor substrate is glycosylated, the desired oligosaccharide moiety is formed. The desired oligosaccharide moiety is one that imparts the desired biological activity upon the glycoprotein or glycolipid to which it is attached. In the compositions of the invention, the preselected saccharide residue is linked to at least about 30% of the potential acceptor sites of interest. More preferably, the preselected saccharide residue is linked to at least about 50% of the potential acceptor substrates of interest, and still more preferably to at least 70% of the potential acceptor substrates of interest. In situations in which the starting glycoprotein or glycolipid exhibits heterogeneity in the oligosaccharide moiety of interest (e.g., some of the oligosaccharides on the starting glycoprotein or glycolipid already have the preselected saccharide residue attached to the acceptor substrate of interest), the recited percentages include such pre-attached saccharide residues.

The term “altered” refers to the glycoprotein or glycolipid of interest having a glycosylation pattern that, after application of the sialyltransferase polypeptides comprising sialyltransferase motifs A and B and methods of the invention, is different from that observed on the glycoprotein as originally produced. An example of such glycoconjugates are glycoproteins in which the glycoforms of the glycoproteins are different from those found on the glycoprotein when it is produced by cells of the organism to which the glycoprotein is native. Also provided are sialyltransferase polypeptides comprising sialyltransferase motifs A and B and methods of using such proteins for enzymatically synthesizing glycoproteins and glycolipids in which the glycosylation pattern of these glycoconjugates are modified compared to the glycosylation pattern of the glycoconjugates as originally produced by a host cell, which can be of the same or a different species than the cells from which the native glycoconjugates are produced.

One can assess differences in glycosylation patterns not only by structural analysis of the glycoproteins and glycolipids, but also by comparison of one or more biological activities of the glycoconjugates. For example, a glycoprotein having an “altered glycoform” includes one that exhibits an improvement in one more biological activities of the glycoprotein after the glycosylation reaction compared to the unmodified glycoprotein. For example, an altered glycoconjugate includes one that, after application of the sialyltransferase polypeptides comprising sialyltransferase motifs A and B and methods of the invention, exhibits a greater binding affinity for a ligand or receptor of interest, a greater therapeutic half-life, reduced antigenicity, and targeting to specific tissues. The amount of improvement observed is preferably statistically significant, and is more preferably at least about a 25% improvement, and still more preferably is at least about 30%, 40%, 50%, 60%, 70%, and even still more preferably is at least 80%, 90%, or 95%.

The products produced using sialyltransferase polypeptides comprising sialyltransferase motifs A and B can be used without purification. However, standard, well known techniques, for example, thin or thick layer chromatography, ion exchange chromatography, or membrane filtration can be used for recovery of glycosylated saccharides. Also, for example, membrane filtration, utilizing a nanofiltration or reverse osmotic membrane as described in commonly assigned AU Patent No. 735695 may be used. As a further example, membrane filtration wherein the membranes have a molecular weight cutoff of about 1000 to about 10,000 Daltons can be used to remove proteins. As another example, nanofiltration or reverse osmosis can then be used to remove salts. Nanofilter membranes are a class of reverse osmosis membranes which pass monovalent salts but retain polyvalent salts and uncharged solutes larger than about 200 to about 1000 Daltons, depending upon the membrane used. Thus, for example, the oligosaccharides produced by the compositions and methods of the present invention can be retained in the membrane and contaminating salts will pass through.

### X. Multienzyme Oligosaccharide Synthesis

As discussed above, in some embodiments, two or more enzymes may be used to form a desired oligosaccharide, including an oligosaccharide determinant on a glycoprotein or glycolipid. For example, a particular oligosaccharide determinant might require addition of a galactose, a sialic acid, and a fucose in order to exhibit a desired activity. Accordingly, the invention provides methods in which two or more glycosyltransferases, e.g., a sialyltransferase polypeptide comprising sialyltransferase motifs A and B, and another glycosyltransferase, such as a fucosyltransferase or a galactosyltransferase, are used to obtain high-yield synthesis of a desired oligosaccharide determinant.

The sialyltransferase polypeptides comprising sialyltransferase motifs A and B prepared as described herein can be used in combination with a multitude of glycosyltransferases. For example, one can use a combination of recombinant sialyltransferase polypeptides comprising sialyltransferase motifs A and B and a recombinant fucosyltranferases, e.g., an H. pylori α1,3/4-fucosyltransferase. For example fucosyltransferases from Helicobacter pylori are disclosed in U.S. Pat. Nos. 6,534,298 and 6,238,894; WO2004009838, published Jan. 29, 2004; U.S. Ser. No. 10/764,212, filed Jan. 22, 2004; each of which are herein incorporated by reference for all purposes. Bacterial glycosyltransferases, including α2,3-sialyltransferases, bifunctional α2,3-2,8-sialyltransferases, β-1,4-GalNActransferases and β-1,3-Galactosyltransferases have been isolated from Campylobacter jejuni and are disclosed in U.S. Pat. No. 6,699,705, issued Mar. 2, 2004, herein incorporated by reference for all purposes. Similarly, the recombinant glycosyltransferases can be used with recombinant accessory enzymes, which may or may not be fused to the glycosyltransferase thereby forming a fusion protein. In other embodiments, the sialyltransferase polypeptides comprising sialyltransferase motifs A and B and additional glycosyltransferases or accessory enzymes are produced in the same cell and used to synthesize a desired end product.

In some cases, a glycoprotein- or glycolipid linked oligosaccharide will include an acceptor substrate for the particular glycosyltransferase of interest upon in vivo biosynthesis of the glycoprotein or glycolipid. Such glycoproteins or glycolipids can be glycosylated using the recombinant glycosyltransferase fusion proteins and methods of the invention without prior modification of the glycosylation pattern of the glycoprotein or glycolipid, respectively. In other cases, however, a glycoprotein or glycolipid of interest will lack a suitable acceptor substrate. In such cases, the methods of the invention can be used to alter the glycosylation pattern of the glycoprotein or glycolipid so that the glycoprotein- or glycolipid-linked oligosaccharides then include an acceptor substrate for the glycosyltransferase-catalyzed attachment of a preselected saccharide unit of interest to form a desired oligosaccharide moiety.

Glycoprotein- or glycolipid linked oligosaccharides optionally can be first “trimmed,” either in whole or in part, to expose either an acceptor substrate for the glycosyltransferase or a moiety to which one or more appropriate residues can be added to obtain a suitable acceptor substrate. Enzymes such as glycosyltransferases and endoglycosidases are useful for the attaching and trimming reactions. For example, a glycoprotein that displays “high mannose”-type oligosaccharides can be subjected to trimming by a mannosidase to obtain an acceptor substrate that, upon attachment of one or more preselected saccharide units, forms the desired oligosaccharide determinant.

The methods are also useful for synthesizing a desired oligosaccharide moiety on a protein or lipid that is unglycosylated in its native form. A suitable acceptor substrate for the corresponding glycosyltransferase can be attached to such proteins or lipids prior to glycosylation using the methods of the present invention. See, e.g., U.S. Pat. No. 5,272,066 for methods of obtaining polypeptides having suitable acceptors for glycosylation.

Thus, in some embodiments, the invention provides methods for in vitro sialylation of saccharide groups present on a glycoconjugate that first involves modifying the glycoconjugate to create a suitable acceptor.

### XI. Uses of Sialyltransferase Polypeptides Comprising Conserved Sequence Motifs and their Sialylated Products

The invention provides sialyltransferase polypeptides comprising sialyltransferase motifs A and B and methods of using the sialyltransferase polypeptides comprising sialyltransferase motifs A and B to enzymatically synthesize glycoproteins, glycolipids, and oligosaccharide moieties. The glycosyltransferase reactions of the invention can take place in vitro in a reaction medium comprising at least one sialyltransferase polypeptide comprising sialyltransferase motifs A and B, acceptor substrate, and donor substrate, and typically a soluble divalent metal cation; or the glycosyltransferase reactions of the invention can take place in vivo. In some embodiments, accessory enzymes and substrates for the accessory enzyme catalytic moiety are also present, so that the accessory enzymes can synthesize the donor substrate for the sialyltransferase polypeptide comprising sialyltransferase motifs A and B.

Product saccharides that can be produced using the methods and reaction mixtures of the invention and are of particular interest include, but are not limited to:

A. Oligosaccharides

The reaction mixtures and methods are useful for producing a wide range of oligosaccharides, including sialyllactose, sialyl-LNnT (LSTd), sialyl-LNT, STn-antigen, and glycosides thereof. The glycosides can include incorporation of linker arms or the like for coupling to other materials.

Among the compounds that one can produce using the recombinant cells, reaction mixtures, and methods of the invention are sialic acid and any sugar having a sialic acid moiety. These include the sialyl galactosides, including the sialyl lactosides, as well as compounds having the formula:

NeuAcα(2→3)Galβ(1→4)GlcN(R′)β-OR or

NeuAcα(2→3)Galβ(1→4)GlcN(R′)β(1→3)Galβ-OR

In these formulae, R′ is alkyl or acyl from 1-18 carbons, 5,6,7,8-tetrahydro-2-naphthamido; benzamido; 2-naphthamido; 4-aminobenzamido; or 4-nitrobenzamido. R is a hydrogen, a alkyl C₁-C₆, a saccharide, an oligosaccharide or an aglycon group having at least one carbon atom. The term “aglycon group having at least one carbon atom” refers to a group -A-Z, in which A represents an alkylene group of from 1 to 18 carbon atoms optionally substituted with halogen, thiol, hydroxy, oxygen, sulfur, amino, imino, or alkoxy; and Z is hydrogen, —OH, —SH, —NH₂, —NHR¹, —N(R¹)₂, —CO₂H, —CO₂R¹, —CONH₂, —CONHR¹, —CON(R¹)₂, —CONHNH₂, or —OR¹ wherein each R¹ is independently alkyl of from 1 to 5 carbon atoms. In addition, R can be (CH₂)ₙCH(CH₂)ₘCH₃

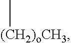

where n,m,o=1-18; (CH₂)ₙ—R² (in which n=0-18), wherein R² is a variously substituted aromatic ring, preferably, a phenyl group, being substituted with one or more alkoxy groups, preferably methoxy or O(CH₂)ₘCH₃, (in which m=0-18), or a combination thereof. R can also be 3-(3,4,5-trimethoxyphenyl)propyl.

A related set of structures included in the general formula are those in which Gal is linked β1,3 and Fuc is linked α11,4. For instance, the tetrasaccharide, NeuAcα2,3Galβ1,3(Fucα4)GlcNAcβ1-, termed here SLe^{a}, is recognized by selectin receptors. See, Berg et al., J. Biol. Chem., 266:14869-14872 (1991). In particular, Berg et al. showed that cells transformed with E-selectin cDNA selectively bound neoglycoproteins comprising SLe^{a}.

The methods of the invention are also useful for synthesizing oligosaccharide compounds having the general formula Galα1,3Gal-, including Galα1,3Galβ1,4Glc(R)β-O—R¹, wherein R¹ is —(CH₂)ₙ—COX, with X═OH, OR², —NHNH₂, R═OH or NAc, and R² is a hydrogen, a saccharide, an oligosaccharide or an aglycon group having at least one carbon atom, and n=an integer from 2 to 18, more preferably from 2 to 10. Also among the compounds that can be synthesized according to the invention are lacto-N-neotetraose (LNnT), GlcNAcβ1,3Galβ1,4Glc (LNT-2), sialyl(α2,3)-lactose, and sialyl(α2,6)-lactose.

The oligosacchrides can be made using sialyltransferase polypeptides comprising sialyltransferase motifs A and B in in vitro reaction mixtures or in fermentative growth of an appropriate recombinant microorganism, as described above.

The recombinant cells, e.g., microorganisms, and reaction mixtures of the invention are particularly useful in synthesizing product saccharides that require multiple enzymatic steps. In these embodiments, the a recombinant cell can contain two or more exogenous glycosyltransferase genes, and produce both of the respective nucleotide sugar substrates. The recombinant cell can then be used form fermentative growth and production of oligosaccharides or can be permeabilized or used for purification of the glycosyltransferases. Alternatively, a reaction mixture can contain two or more types of recombinant cells, each of which contains one or more exogenous glycosyltransferase genes and the corresponding nucleotide sugar generating system. For example, one can use a combination of recombinant cell types, one of which contains an exogenous sialyltransferase gene and a system for producing CMP-sialic acid, and another recombinant cell type that contains an exogenous galactosyltransferase gene and produces UDP-Gal. In this group of embodiments, the different cell types can be combined in an initial reaction mixture, or preferably the recombinant cell types for a second glycosyltransferase reaction can be added to the reaction medium once the first glycosyltransferase reaction has neared completion. By conducting two glycosyltransferase reactions in sequence in a single vessel, overall yields are improved over procedures in which an intermediate species is isolated. Moreover, cleanup and disposal of extra solvents and by-products is reduced.

For example, the present invention provides recombinant cells and methods for the preparation of compounds having the formula:

NeuAcα(2→3)Galβ(1→4)(Fucα1→3)GlcN(R′)β(1→3)Galβ-OR

In this formula, R is a hydrogen, a saccharide, an oligosaccharide or an aglycon group having at least one carbon atom. R′ can be either acetyl or allyloxycarbonyl (Alloc).

The term “aglycon group having at least one carbon atom” refers to a group -A-Z, in which A represents an alkylene group of from 1 to 18 carbon atoms optionally substituted with halogen, thiol, hydroxy, oxygen, sulfur, amino, imino, or alkoxy; and Z is hydrogen, —OH, —SH, —NH₂, —NHR¹, —N(R¹)₂, —CO₂H, —CO₂R¹, —CONH₂, —CONHR¹, —CON(R¹)₂, —CONHNH₂, or —OR¹ wherein each R¹ is independently alkyl of from 1 to 5 carbon atoms. In addition, R can be (CH₂)ₙCH(CH₂)ₘCH₃

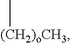

where n,m,o=1-18; (CH₂)ₙ—R² (in which n=0-18), wherein R² is a variously substituted aromatic ring, preferably, a phenyl group, being substituted with one or more alkoxy groups, preferably methoxy or O(CH₂)ₘCH₃, (in which m=0-18), or a combination thereof.

The steps involved in synthesizing these compounds include:
(a) galactosylating a compound of the formula GlcNR′β(1→3)Galβ-OR with a galactosyltransferase in the presence of a UDP-galactose under conditions sufficient to form the compound: Galβ(1→4)GlcNR′β(1→3)Galβ-OR;
(b) sialylating the compound formed in (a) with a sialyltransferase in the presence of a CMP derivative of a sialic acid using a α(2,3)sialyltransferase under conditions in which sialic acid is transferred to the non-reducing sugar to form the compound: NeuAcα(2→3)Galβ(1→4)GlcNR′β(1→3)Galβ-OR; and
(c) fucosylating the compound formed in (b) to provide the NeuAcα(2→3)Galβ(1→4)(Fucα1→3)GlcNR′β(1→3)Galβ-OR.

The recombinant cells of the invention provide an efficient way to carry out each of these steps, either individually or simultaneously. One or more of the steps can be conducted using the recombinant cells of the invention. For example, the sialylation and galactosylation reaction can be accomplished using a recombinant cell disclosed herein, that also contains an exogenous galactosyltransferase gene and which produces UDP-Gal. The fucosylating steps can also be carried out using recombinant cells that produce the appropriate glycosyltransferase and donor sugar, or can be carried out using conventional non-cell-based methods.

In one embodiment, R is ethyl, the fucosylation step is carried out chemically, and the galactosylation and sialylation steps are carried out in a cell as disclosed herein.

In some embodiments, the recombinant cells and reaction mixtures are constructed for production of a sialylated saccharide product that is also fucosylated. Through use of a cell that produces GDP-fucose and contains the appropriate fucosyltransferase enzymes, the following carbohydrate structures are among those that one can obtain: (1) Fucα(1→2) Galβ-; (2) Galβ(1→3)[Fucα(1→4)]GlcNAcβ-; (3) Galβ(1→4) [Fucα(1→3)]GlcNAcβ-; (4) Galβ(1→4)[Fucα(1→3)]Glc; (5)-GlcNAcβ(1→4) [Fucα(1→6)]GlcNAcβ1→Asn; (6)-GlcNAcβ(1→4)[Fucα(1→3)GlcNAcβ1→Asn; (7) Fucα(1→6)Galβ→; (8) Fucα(1→3) Galβ-; (9) Glcβ(1→3)Fucα1→O-Thr and Fucα1→O-Thr/Ser; and (10) Fucα1→Ceramide. Examples of sialylated products that can be formed using GDP-fucose as a reactant include, but are not limited to, 3′—Sialyl-3-fucosyllactose, Sialyl lewis X, and Sialyl lewis A.

Galactosylated/sialylated products can also be produced using the recombinant cells and methods of the invention. For example, by use of a recombinant cell that produces UDP-Gal and contains the appropriate galactosyltransferase, one can add Gal in a β1,4 linkage, an α1,3 linkage, an α1,4 linkage, or a β1,3 linkage to a saccharide that includes a GlcNAc or Glc residue. The recombinant cells are permeabilized and placed in contact with the acceptor saccharide, resulting of transfer of the Gal from the UDP-Gal to the acceptor. One example of such an oligosaccharide for which the invention provides an efficient method of synthesis is lacto-N-neotetraose, Galβ(1-4)-GlcNAcβ(1-3)-Galβ(1-4)-Glc (formula I). See, e.g., Min-Yuan Chou et al. (1996) J. Biol. Chem. 271 (32): 19166-19173.

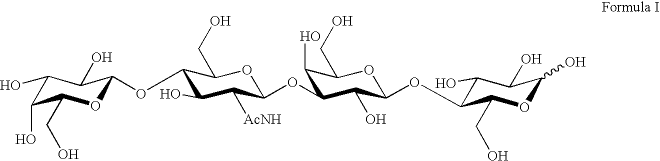

Sialylated products comprising GlcNAc or GalNAc residues can also be produced. The invention also provides methods for adding GalNAc or GlcNAc to Gal, in a β1,3 linkage or a β1,4 linkage, by providing a recombinant cell disclosed herein that encodes a GalNAc transferase or GlcNAc transferase and which produces an activated UDP-GalNAc or UDP-GlcNAc.

In the above descriptions, the terms are generally used according to their standard meanings. The term “alkyl” as used herein means a branched or unbranched, saturated or unsaturated, monovalent or divalent, hydrocarbon radical having from 1 to 20 carbons, including lower alkyls of 1-8 carbons such as methyl, ethyl, n-propyl, butyl, n-hexyl, and the like, cycloalkyls (3-7 carbons), cycloalkylmethyls (4-8 carbons), and arylalkyls. The term “alkoxy” refers to alkyl radicals attached to the remainder of the molecule by an oxygen, e.g., ethoxy, methoxy, or n-propoxy. The term “alkylthio” refers to alkyl radicals attached to the remainder of the molecule by a sulfur. The term of “acyl” refers to a radical derived from an organic acid by the removal of the hydroxyl group. Examples include acetyl, propionyl, oleoyl, myristoyl.

The term “aryl” refers to a radical derived from an aromatic hydrocarbon by the removal of one atom, e.g., phenyl from benzene. The aromatic hydrocarbon may have more than one unsaturated carbon ring, e.g., naphthyl.

The term “alkoxy” refers to alkyl radicals attached to the remainder of the molecule by an oxygen, e.g., ethoxy, methoxy, or n-propoxy.

The term “alkylthio” refers to alkyl radicals attached to the remainder of the molecule by a sulfur.

An “alkanoamido” radical has the general formula —NH—CO—(C₁-C₆ alkyl) and may or may not be substituted. If substituted, the substituent is typically hydroxyl. The term specifically includes two preferred structures, acetamido, —NH—CO—CH₃, and hydroxyacetamido, —NH—CO—CH₂—OH.

The term “heterocyclic compounds” refers to ring compounds having three or more atoms in which at least one of the atoms is other than carbon (e.g. N, O, S, Se, P, or As). Examples of such compounds include furans (including the furanose form of pentoses, such as fucose), pyrans (including the pyranose form of hexoses, such as glucose and galactose) pyrimidines, purines, pyrazines and the like.

A list of structure comprised within sialylated products follows in Table 2. Each of the oligosaccharides listed below can be synthesized as an unconjugated product, or can by conjugated to, e.g., a glycolipid or a glycoprotein or a glycopeptide. Those of skill will recognize that the list is incomplete and that variations of these structures can also be synthesized.

TABLE 2
Oligosaccharide Formulas and Enzyme Activities Needed

| | Enzymes that can be |
|---|---|
| Structure | used for synthesis |
| Siaα2-3Galβ1-4Glc | A, I |
| Siaα2-6Galβ1-4Glc | A, J |
| Siaα2-3Galβ1-4GlcNAc | A, I |
| Siaα2-6Galβ1-4GlcNAc | A, J |
| Siaα2-3Galβ1-4(Fucα1-3)Glc | A, H, I |
| Siaα2-3Galβ1-3GlcNAcβ1-3Galβ1-4Glc | A, E, I |
| Galβ1-3(Siaα2-6)GlcNAcβ1-3Galβ1-4Glc | A, B, E, J |
| Siaα2-6Galβ1-4 GlcNAcβ1-3Galβ1-4Glc | A, B, E, J |
| Siaα2-3Galβ1-4 GlcNAcβ1-3Galβ1-4Glc | A, B, E, I |
| Siaα2-3(Siaα2-6)Galβ1-4 GlcNAcβ1- | A, B, E, I, J |
| 3Galβ1-4Glc | |
| Siaα2-3Galβ1-4(Fucα1-3)GlcNAc | A, H, I |
| Siaα2-3Galβ1-3(Fucα1-4)GlcNAc | B, H, I |
| Siaα2-3Galβ1-3GalNAcβ1-4Galα1-4Galβ1- | A, B, C, F, G, I |
| 4Glc | |
| Siaα2-3Galβ1-3GalNAcβ1-3Galα1-3Galβ1- | A, B, D, F, I |
| 4Glc | |
| Siaα2-3Galβ1-3GalNAcβ1-4Galβ1-4Glc | A, B, F, I |
| Siaα2-3Galβ1-3(Siaα2-6)GalNAcβ1- | A, B, F, I, J |
| 4Galβ1-4Glc | |
| Siaα2-3Galβ1-3(Siaα2-8Siaα2- | A, B, F, I, J, K |
| 6)GalNAcβ1-4Galβ1-4Glc | |
| Siaα2-8Siaα2-3Galβ1-3(Siaα2-8Siaα2- | A, B, F, I, J, K |
| 6)GalNAcβ1-4Galβ1-4Glc | |
| GalNAcβ1-4(Siaα2-3)Galβ1-4Glc | A, F, I |
| Galβ1-3GalNAcβ1-4(Siaα2-3)Galβ1-4Glc | A, B, F, I |
| Siaα2-3Galβ1-3GalNAcβ1-4(Siaα2- | A, B, F, I |
| 3)Galβ1-4Glc | |
| Siaα2-8Siaα2-3Galβ1-3GalNAcβ1-4(Siaα2- | A, B, F, I, K |
| 3)Galβ1-4Glc | |
| Siaα2-8Siaα2-3Galβ1-4Glc | A, I, K |
| GalNAcβ1-4(Siaα2-8Siaα2-3)Galβ1-4Glc | A, F, I, K |
| Galβ1-3GalNAcβ1-4(Siaα2-8Siaα2- | A, B, F, I, K |
| 3)Galβ1-4Glc | |
| Siaα2-3Galβ1-3 GalNAcβ1-4(Siaα2-8Siaα2- | A, B, F, I, K |
| 3)Gal β1-4Glc | |
| Siaα2-8Siaα2-3Galβ1-3GalNAcβ1-4(Siaα2- | A, B, F, I, K |
| 8Siaα2-3)Galβ1-4Glc | |
| Siaα2-8Siaα2-8Siaα2-3Galβ1-4Glc | A, I, K |
| GalNAcβ1-4(Siaα2-8Siaα2-8Siaα2- | A, F, I, K |
| 3)Galβ1-4Glc | |
| Galβ1-3GalNAcβ1-4(Siaα2-8Siaα2-8Siaα2- | A, B, F, I, K |
| 3)Galβ1-4Glc | |
| Siaα2-3Galβ1-3GalNAcβ1-4(Siaα2-8Siaα2- | A, B, F, I, K |
| 8Siaα2-3)Galβ1-4Glc | |
| Fucα1-2Galβ1-3GalNAcβ1-4(Siaα2- | A, B, F, G, I |
| 3)Galβ1-4Glc | |

Key:
A = β1,4Galactosyltransferase (e.g., lgtB- Neisseria meningitidis/gonorrhoeae)
B = β1,3Galactsoyltransferase (e.g., cgtB- C. jejuni)
C = α1,4Galactosyltraferase (e.g., lgtC- Neisseria meningitidis/gonorrhoeae)
D = α1,3Galactosaminyltransferase (e.g., mouse or bovine enzyme)
E = β1,3N-actylglucosaminyltransferase (e.g., lgtA-Neisseria meningitidis/gonorrhoeae)
F = β1,4N-acetylgalactosaminyltransferase (e.g., cgtA-C. jejuni)
G = α1,2Fucosyltransferase (e.g., futC-H. pylori)
H = α1,3/4Fucosyltransferase (e.g., futA/b-H. pylori)
I = α2,3Sialyltransferase
J = α2,6Sialyltransferase
K = α2,8Sialyltransferase

B. Glycolipids, Including Gangliosides and Related Structures

The reaction mixtures and cells of the invention are also useful for producing many different glycolipids. Those of particular interest include, for example, lactosylceramide, glucosylceramide, Globo-H, Globotetrose, lipopolysaccharides and various forms of these lipids. For example, the lipids can be modified to be, for example, a lyso-, deacetyl, linker arm-containing, or an O-acetyl forms.

The invention provides reaction mixtures, cell types, and methods for adding one or more saccharide moieties in a specific manner in order to obtain a desired ganglioside or other glycosphingolipid, or derivatives thereof. The methods of the invention involve the use of cells that express one or more recombinant glycosyltransferases to synthesize glycosphingoids, including gangliosides and other glycosphingoids. Through use of a glycosyltransferase to link a desired carbohydrate to the precursor molecule, one can achieve a desired linkage with high specificity. In some embodiments, it is desirable to remove the fatty acid moiety from the sphingoid precursor prior to the glycosyltransferase reaction, and/or to use an organic solvent to facilitate the reaction. Enzymes and reaction schemes for producing many gangliosides and related structures are described in PCT Patent Application No. PCT/US/25470, which was published on Jun. 10, 1999 as Publication No. WO99/28491 and is entitled “Enzymatic synthesis of gangliosides.”

The methods of the invention are useful for producing any of a large number of gangliosides and related structures. Many gangliosides of interest are described in Oettgen, H. F., ed., Gangliosides and Cancer, VCH, Germany, 1989, pp. 10-15, and references cited therein. Gangliosides of particular interest include, for example, those found in the brain as well as other sources which are listed in Table 3.

TABLE 3
Ganglioside Formulas and Abbreviations

| Structure | Abbreviation |
|---|---|
| Neu5Ac3Gal4GlcCer | GM3 |
| GalNAc4(Neu5Ac3)Gal4GlcCer | GM2 |
| Gal3GalNAc4(Neu5Ac3)Gal4GlcCer | GM1a |
| Neu5Ac3Gal3GalNAc4Gal4GlcCer | GM1b |
| Neu5Ac8Neu5Ac3Gal4GlcCer | GD3 |
| GalNAc4(Neu5Ac8Neu5Ac3)Gal4GlcCer | GD2 |
| Neu5Ac3Gal3GalNAc4(Neu5Ac3)Gal4GlcCer | GD1a |
| Neu5Ac3Gal3(Neu5Ac6)GalNAc4Gal4GlcCer | GD1α |
| Gal3GalNAc4(Neu5Ac8Neu5Ac3)Gal4GlcCer | GD1b |
| Neu5Ac8Neu5Ac3Gal3GalNAc4(Neu5Ac3)Gal4GlcCer | GT1a |
| Neu5Ac3Gal3GalNAc4(Neu5Ac8Neu5Ac3)Gal4GlcCer | GT1b |
| Gal3GalNAc4(Neu5Ac8Neu5Ac8Neu5Ac3)Gal4GlcCer | GT1c |
| Neu5Ac8Neu5Ac3Gal3GalNAc4(Neu5Ac8Neu5c3)Gal4GlcCer | GQ1b |

Nomenclature of Glycolipids, IUPAC-IUB Joint Commission on Biochemical Nomenclature (Recommendations 1997); Pure Appl. Chem. (1997) 69: 2475-2487; Eur. J. Biochem (1998) 257: 293-298) (www.chem.qmw.ac.uk/iupac/misc/glylp.html).

C. Glycopeptides and Glycoproteins

In some embodiments, the product saccharides are attached to polypeptides. The sialyltransferase polypeptide comprising sialyltransferase motifs A and B, reaction mixtures, and cells of the invention are thus useful for modifying glycoproteins to achieve various improvements in properties such as therapeutic half-life, immunogenicity, and the like. Examples of glycopeptides of particular interest include, for example, STn-peptide, Tn-peptide, T-peptide, ST-peptide, and the linked versions of these structures. Enzymes and reactions that are useful for modification of glycoproteins are described in, for example, PCT Patent Application No. US98/00835, which was published as WO98/31826 on Jul. 23, 1998.

The sialyltransferase polypeptides comprising sialyltransferase motifs A and B can be used to modify or to synthesize N-linked glycoproteins, i.e., N-linked glycans. For example the sialyltransferase polypeptides comprising sialyltransferase motifs A and B can be used to modify or to synthesize complex type N-linked glycans, e.g., bi-antennary, tri-antennary, tetra- antennary or penta-antennary oligosaccharide structures. The sialyltransferase polypeptides comprising sialyltransferase motifs A and B can be used to modify or to synthesize O-linked glycoproteins.

In some embodiments, the sialyltransferase polypeptides comprising sialyltransferase motifs A and B synthesize a glycoprotein comprising a Sia-α2,6-GalNAc-amino acid structure. The proteins can also be used to synthesize glycoproteins comprising a Sia-α2,3-Gal-β1,3-GalNAc-amino acid structure, or a Sia-α2,3-Gal-β1,4-GlcNAc-amino acid structure, or a Sia-α2,3-Gal-β1,4Glu-amino acid structure. The identity of the amino acid for linkage of the oligosaccharide to the gloycoprotein is not critical and is not limited to Asn, Ser, or Thr.

D. Pharmaceutical and Other Applications

The compounds described above can then be used in a variety of applications, e.g., as antigens, diagnostic reagents, foodstuffs, or as therapeutics. Thus, the present invention also provides pharmaceutical compositions which can be used in treating a variety of conditions. The pharmaceutical compositions are comprised of oligosaccharides made according to the methods described above.

Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions are intended for parenteral, intranasal, topical, oral or local administration, such as by aerosol or transdermally, for prophylactic and/or therapeutic treatment. Commonly, the pharmaceutical compositions are administered parenterally, e.g., intravenously. Thus, the invention provides compositions for parenteral administration which comprise the compound dissolved or suspended in an acceptable carrier, preferably an aqueous carrier, e.g., water, buffered water, saline, PBS and the like. The compositions may contain pharmaceutically acceptable auxiliary substances as required to approximate physiological conditions, such as pH adjusting and buffering agents, tonicity adjusting agents, wetting agents, detergents and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9 and most preferably from 7 and 8.

In some embodiments the oligosaccharides of the invention can be incorporated into liposomes formed from standard vesicle-forming lipids. A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028. The targeting of liposomes using a variety of targeting agents (e.g., the sialyl galactosides of the invention) is well known in the art (see, e.g., U.S. Pat. Nos. 4,957,773 and 4,603,044).

The compositions containing the oligosaccharides can be administered for prophylactic and/or therapeutic treatments. In therapeutic applications, compositions are administered to a patient already suffering from a disease, as described above, in an amount sufficient to cure or at least partially arrest the symptoms of the disease and its complications. An amount adequate to accomplish this is defined as a “therapeutically effective dose.” Amounts effective for this use will depend on the severity of the disease and the weight and general state of the patient, but generally range from about 0.5 mg to about 40 g of oligosaccharide per day for a 70 kg patient, with dosages of from about 5 mg to about 20 g of the compounds per day being more commonly used.

Single or multiple administrations of the compositions can be carried out with dose levels and pattern being selected by the treating physician. In any event, the pharmaceutical formulations should provide a quantity of the oligosaccharides of this invention sufficient to effectively treat the patient.

The oligosaccharides may also find use as diagnostic reagents. For example, labeled compounds can be used to locate areas of inflammation or tumor metastasis in a patient suspected of having an inflammation. For this use, the compounds can be labeled with appropriate radioisotopes, for example, ¹²⁵I, ¹⁴C, or tritium.

The oligosaccharide of the invention can be used as an immunogen for the production of monoclonal or polyclonal antibodies specifically reactive with the compounds of the invention. The multitude of techniques available to those skilled in the art for production and manipulation of various immunoglobulin molecules can be used in the present invention. Antibodies may be produced by a variety of means well known to those of skill in the art.

The production of non-human monoclonal antibodies, e.g., murine, lagomorpha, equine, etc., is well known and may be accomplished by, for example, immunizing the animal with a preparation containing the oligosaccharide of the invention. Antibody-producing cells obtained from the immunized animals are immortalized and screened, or screened first for the production of the desired antibody and then immortalized. For a discussion of general procedures of monoclonal antibody production, see, Harlow and Lane, Antibodies, A Laboratory Manual, Cold Spring Harbor Publications, N.Y. (1988).

### XII. Conjugation of Modified Sugars to Peptides

The modified sugars are conjugated to a glycosylated or non-glycosylated peptide or protein using an appropriate enzyme to mediate the conjugation. Preferably, the concentrations of the modified donor sugar(s), enzyme(s) and acceptor peptide(s) or protein(s) are selected such that glycosylation proceeds until the acceptor is consumed.

A number of methods of using glycosyltransferases to synthesize desired oligosaccharide structures are known and are generally applicable to the instant invention. Exemplary methods are described, for instance, WO 96/32491, Ito et al., Pure Appl. Chem. 65: 753 (1993), and U.S. Pat. Nos. 5,352,670, 5,374,541, and 5,545,553.

In a some embodiments, an endoglycosidase is used in the reaction in combination with glycosyltransferases. The enzymes are used to alter a saccharide structure on the peptide at any point either before or after the addition of the modified sugar to the peptide.

In another embodiment, the method makes use of one or more exo- or endoglycosidase. The glycosidase is typically a mutant, which is engineered to form glycosyl bonds rather than rupture them. The mutant glycanase typically includes a substitution of an amino acid residue for an active site acidic amino acid residue. For example, when the endoglycanase is endo-H, the substituted active site residues will typically be Asp at position 130, Glu at position 132 or a combination thereof. The amino acids are generally replaced with serine, alanine, asparagine, or glutamine.

The mutant enzyme catalyzes the reaction, usually by a synthesis step that is analogous to the reverse reaction of the endoglycanase hydrolysis step. In these embodiments, the glycosyl donor molecule (e.g., a desired oligo- or mono-saccharide structure) contains a leaving group and the reaction proceeds with the addition of the donor molecule to a GlcNAc residue on the protein. For example, the leaving group can be a halogen, such as fluoride. In other embodiments, the leaving group is a Asn, or a Asn-peptide moiety. In yet further embodiments, the GlcNAc residue on the glycosyl donor molecule is modified. For example, the GlcNAc residue may comprise a 1,2 oxazoline moiety.

In a preferred embodiment, each of the enzymes utilized to produce a conjugate of the invention are present in a catalytic amount. The catalytic amount of a particular enzyme varies according to the concentration of that enzyme's substrate as well as to reaction conditions such as temperature, time and pH value. Means for determining the catalytic amount for a given enzyme under preselected substrate concentrations and reaction conditions are well known to those of skill in the art.

The temperature at which an above process is carried out can range from just above freezing to the temperature at which the most sensitive enzyme denatures. Preferred temperature ranges are about 0° C. to about 55° C., and more preferably about 20° C. to about 30° C. In another exemplary embodiment, one or more components of the present method are conducted at an elevated temperature using a thermophilic enzyme.

The reaction mixture is maintained for a period of time sufficient for the acceptor to be glycosylated, thereby forming the desired conjugate. Some of the conjugate can often be detected after a few hours, with recoverable amounts usually being obtained within 24 hours or less. Those of skill in the art understand that the rate of reaction is dependent on a number of variable factors (e.g, enzyme concentration, donor concentration, acceptor concentration, temperature, solvent volume), which are optimized for a selected system.

The present invention also provides for the industrial-scale production of modified peptides. As used herein, an industrial scale generally produces at least one gram of finished, purified conjugate.

In the discussion that follows, the invention is exemplified by the conjugation of modified sialic acid moieties to a glycosylated peptide using sialyltransferase polypeptides comprising sialyltransferase motifs A and B. The exemplary modified sialic acid is labeled with PEG. The focus of the following discussion on the use of PEG-modified sialic acid and glycosylated peptides is for clarity of illustration and is not intended to imply that the invention is limited to the conjugation of these two partners. Moreover, the discussion is equally applicable to the modification of a glycosyl unit with agents other than PEG including other water-soluble polymers, therapeutic moieties, and biomolecules.

An enzymatic approach can be used for the selective introduction of PEGylated or PPGylated carbohydrates onto a peptide or glycopeptide. The method utilizes modified sugars containing PEG, PPG, or a masked reactive functional group, and is combined with the appropriate glycosyltransferase or glycosynthase. By selecting the glycosyltransferase that will make the desired carbohydrate linkage and utilizing the modified sugar as the donor substrate, the PEG or PPG can be introduced directly onto the peptide backbone, onto existing sugar residues of a glycopeptide or onto sugar residues that have been added to a peptide.

An acceptor for the sialyltransferase is present on the peptide to be modified by the methods of the present invention either as a naturally occurring structure or one placed there recombinantly, enzymatically or chemically. Suitable acceptors, include, for example, galactosyl acceptors such as Galβ1,4GlcNAc, Galβ1,4GalNAc, Galβ1,3GalNAc, lacto-N-tetraose, Galβ1,3GlcNAc, Galβ1,3Ara, Galβ1,6GlcNAc, Galβ1,4Glc (lactose), and other acceptors known to those of skill in the art (see, e.g., Paulson et al., J. Biol. Chem. 253: 5617-5624 (1978)).

In one embodiment, an acceptor for the sialyltransferase is present on the glycopeptide to be modified upon in vivo synthesis of the glycopeptide. Such glycopeptides can be sialylated using the claimed methods without prior modification of the glycosylation pattern of the glycopeptide. Alternatively, the methods of the invention can be used to sialylate a peptide that does not include a suitable acceptor; one first modifies the peptide to include an acceptor by methods known to those of skill in the art. In an exemplary embodiment, a GalNAc residue is added by the action of a GalNAc transferase.

In an exemplary embodiment, the galactosyl acceptor is assembled by attaching a galactose residue to an appropriate acceptor linked to the peptide, e.g., a GlcNAc. The method includes incubating the peptide to be modified with a reaction mixture that contains a suitable amount of a galactosyltransferase (e.g., galβ1,3 or galβ1,4), and a suitable galactosyl donor (e.g., UDP-galactose). The reaction is allowed to proceed substantially to completion or, alternatively, the reaction is terminated when a preselected amount of the galactose residue is added. Other methods of assembling a selected saccharide acceptor will be apparent to those of skill in the art.

In yet another embodiment, glycopeptide-linked oligosaccharides are first “trimmed,” either in whole or in part, to expose either an acceptor for the sialyltransferase or a moiety to which one or more appropriate residues can be added to obtain a suitable acceptor. Enzymes such as glycosyltransferases and endoglycosidases (see, for example U.S. Pat. No. 5,716,812) are useful for the attaching and trimming reactions.

Methods for conjugation of modified sugars to peptides or proteins are found e.g., in U.S. Ser. No. 60/328,523 filed Oct. 10, 2001; U.S. Ser. No. 60/387,292, filed Jun. 7, 2002; U.S. Ser. No. 60/391,777 filed Jun. 25, 2002; U.S. Ser. No. 60/404,249 filed Aug. 16, 2002; PCT/US02/32263; US Published Patent application 20040142856, filed Apr. 9, 2003, and published Jul. 22, 2004; US Published Patent application 20040137557, filed Nov. 5, 2002, 2003, and published Jul. 15, 2004; US Published Patent application 20040132640, filed Apr. 9, 2003, and published Jul. 8, 2004; US Published Patent application 20040126838, filed Apr. 9, 2003, and published Jul. 1, 2004; US Published Patent application 20040115168, filed Apr. 9, 2003, and published Jun. 17, 2004; US Published Patent application 20040082026, filed Apr. 9, 2003, and published Apr. 29, 2004; US Published Patent application 20040077836, filed Apr. 9, 2003, and published Apr. 22, 2004; US Published Patent application 20040063911, filed Apr. 9, 2003, and published Apr. 1, 2004; and US Published Patent application 20040043446, filed Apr. 9, 2003, and published Mar. 4, 2004; each of which are herein incorporated by reference for all purposes.

It must be noted that as used herein and in the appended claims, the singular forms “a”, “and”, and “the” include plural referents unless the context clearly dictates otherwise. Thus, for example, reference to “a nucleic acid” includes a plurality of such nucleic acids and reference to “the polypeptide” includes reference to one or more polypeptides and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. Citations are incorporated herein by reference.

### EXAMPLES

### Example 1

### Identification of Cst-I Enzymes in Campylobacter jejuni Strains O:19 and O:36

Cloning the Cst-I nucleic acids. Genomic DNA was isolated from C. jejuni strain O:19 and from C. jejuni strain O:36. PCR was performed using primers CJ18F and CJ40R under stringent conditions. Nucleic acid sequences and encoded amino acid sequences are shown in FIGS. 2 and 3.

Results. Nucleic acids encoding Cst-I enzymes were isolated from C. jejuni strain O:19 and from C. jejuni strain O:36. Both enzymes comprise sialyltransferase motifs A and B.

### Example 2

### Active Truncations of Cst-I Enzymes from Campylobacter jejuni

Truncations were made of the Cst-I enzyme from C. jejuni strain OH4384, by making appropriate deletions of the nucleic acid encoding the protein. Truncated proteins were expressed as fusions with the MalE protein. A thrombin cleavage site was included between the MalE protein and the Cst-I enzyme to facilitate purification of the truncated protein.

Assays. Protein concentration was determined using the bicinchoninic acid protein assay kit (Pierce, Rockford, Ill.). For all of the enzymatic assays, one unit of activity was defined as the amount of enzyme that generated one mol of product per minute. FCHASE-labelled oligosaccharides are prepared as described in Gilbert et al. (1997) Eur. J. Biochem. 249: 187-194. p-Nitrophenol-glycosides (p-NP-glycosides) were obtained from Sigma-Aldrich.

The -2,3-sialyltransferase activity was assayed at 37° C. using 1 mM Lac-FCHASE (6-(5-fluorescein-carboxamido)-hexanoic acid succimidyl ester), 0.2 mM CMP-Neu5Ac, 50 mM MOPS pH 7, 10 mM MnCl₂ and 10 mM MgCl₂ in a final volume of 10 μL. After 5 min the reaction mixtures with fluorogenic acceptors were diluted with 10 mM NaOH and analyzed by capillary electrophoresis performed using the separation conditions as described previously (Gilbert et al. (1997) supra.).

Kinetic analysis of acceptors was performed at 37° C. with p —NP-glycosides at concentrations of 0.1 to 10 mM, with CMP-Neu5Ac at 1 mM. Kinetic analysis of the donor CMP-Neu5Ac was performed at a concentration of 20 μM to 1000 μM with p-NP-lactose at 5 mM. Care was taken to ensure that the level of acceptor conversion was between about 5-10% for acceptor kinetic assays.

For donor kinetics the amount of conversion of CMP-Neu5Ac was calculated from the amount of product formed compared to an internal standard of 10 μM p-NP-glucose added after the reaction. This peak was well resolved from the acceptor and product peaks. The reactions with p-NP-glycosides were stopped by addition of an equal volume of 2% SDS, 20 mM EDTA and heated to 75° C. for 3 minutes and then diluted 1:1 (or maximally 1:10 for 10 mM concentrations) with water. The samples were then analyzed by CE using a diode array detector scanning between 260 and 300 nm, with the peaks at detected at 290 nm. The peaks from the electropherograms were analyzed using manual peak integration with the P/ACE Station™ software. For rapid detection of enzyme activity, samples from the transferase reaction mixtures were examined by thin layer chromatography on silica-60 TLC plates (E. Merck) as described in Gilbert et al. (1996) supra.

Results: A Cst-I truncation (Cst-95) from strain OH4384 comprising amino acids 1-285 of the full-length, 430 amino acid protein retained activity. The first 285 amino acids of the Cst-1 proteins from strain O:19 are identical to amino acid residues 1-285 of the OH4384 protein. The Cst-1 protein from strain O:36 differs form the OH4384 strain at two residues (i.e., 99 and 283). The Cst-95 protein was expressed in E. coli with yields of about 500 units per liter of bacterial culture.

### Example 3

### Activity of Cst-I Enzymes in Campylobacter jejuni Strains O:19 and O:36

Expression of the Cst-I proteins from C. jejuni strain O:19 and from C. jejuni strain O:36. Nucleic acids encoding Cst-I proteins from C. jejuni strain O:19 and from C. jejuni strain O:36 were cloned into expression vectors for expression in E. coli. E. coli were transformed with the expression vectors, grown under conditions suitable to express the sialyltransferase proteins, harvested, and lysed. Lysates comprising the Cst-I expression products were assayed for sialyltransferase activity as described above and both Cst-I proteins from C. jejuni strain O:19 and from C. jejuni strain O:36 catalyze the transfer of Neu5Ac from CMP-Neu5Ac to an acceptor. The O:19 and O:36 activities were compared to activity of the protein from Cst-I OH4384. The following values were obtained: Cst-I OH4384, 346.2 mU/ml; Cst-I O:19 324.9 mU/ml; and Cst-I O:36, 50.3 mU/ml.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1. An isolated or recombinant sialyltransferase polypeptide;
wherein the sialyltransferase polypeptide transfers sialic acid from a donor substrate to an acceptor substrate; and wherein the sialyltransferase polypeptide comprises a sialyltransferase motif A and a sialyltransferase motif B;
with the proviso that the sialytransferase polypeptide is not a member of the group selected from GenBank AF130466, GenBank AX934425, GenBank AX934434, GenBank AX934427, GenBank AX934431, GenBank AF401529, GenBank AX934436, GenBank AX934429, GenBank AY044156, GenBank AF400047, GenBank AY297047, GenBank AF305571, GenBank AL139077, GenBank X57315, GenBank AE006157, SEQ ID NO: 43 through SEQ ID NO:57 and a consensus sequence of a sialyltransferase protein derived from CD: pfam06002.2, CST-I, (SEQ ID NO:32).

2. The sialyltransferase polypeptide of claim 1, wherein the sialyltransferase motif A is DVFRCNQFYFED/E (SEQ ID NO: 1).

3. The sialyltransferase polypeptide of claim 2, wherein the sialyltransferase motif B is RITSGVYMC (SEQ ID NO:2)

4. The sialyltransferase polypeptide of claim 2, wherein the sialyltransferase motif A is DVFRCNQFYFED (SEQ ID NO:3).

5. The sialyltransferase polypeptide of claim 2, wherein the sialyltransferase motif A is DVFRCNQFYFEE (SEQ ID NO:4).

6. The sialyltransferase polypeptide of claim 1, wherein the sialyltransferase motif B is RITSGVYMC (SEQ ID NO:2).

7. The sialyltransferase polypeptide of claim 1, wherein the sialyltransferase polypeptide has α-2,3-sialyltransferase activity.

8. The sialyltransferase polypeptide of claim 1, wherein the sialyltransferase polypeptide has α-2,8-sialyltransferase activity.

9. The sialyltransferase polypeptide of claim 7, wherein the sialyltransferase polypeptide has α-2,8-sialyltransferase activity.

10. The sialyltransferase polypeptide of claim 1, wherein the acceptor molecule is an oligosaccharide, a glycolipid, a glycopeptide, or a glycoprotein.

11. The sialyltransferase polypeptide of claim 1, wherein the sialyltransferase polypeptide is truncated.

12. The sialyltransferase polypeptide of claim 1, wherein the sialyltransferase polypeptide is a bacterial protein

13. The sialyltransferase polypeptide of claim 12, wherein the sialyltransferase polypeptide is from a member of the family Vibrionaceae.

14. The sialyltransferase polypeptide of claim 12, wherein the sialyltransferase polypeptide is from a bacterial species selected from the group consisting of Haemophilus influenzae, Pasteurella multocida, and Campylobacter species.

15. The sialyltransferase polypeptide of claim 14, wherein the sialyltransferase polypeptide is from Campylobacter jejuni.

16. The sialyltransferase polypeptide of claim 15, wherein the sialyltransferase polypeptide is from strain O:19.

17. The sialyltransferase polypeptide of claim 15, wherein the sialyltransferase polypeptide is from strain O:36.

18. The sialyltransferase polypeptide of claim 1, wherein the sialyltransferase polypeptide further comprises an amino acid tag.

19. The sialyltransferase polypeptide of claim 1, wherein the sialyltransferase polypeptide is fused to an accessory enzyme.

20. An isolated or recombinant sialyltransferase polypeptide;
wherein the sialyltransferase polypeptide transfers sialic acid from a donor substrate to an acceptor substrate; and
wherein the sialyltransferase polypeptide comprises an amino acid sequence with at least 98% identity to the amino acid sequence of FIG. 2 (O:36 amino acid sequence, SEQ ID NO:6).

21. The sialyltransferase polypeptide of claim 20, wherein the sialyltransferase polypeptide has α-2,3-sialyltransferase activity.

22. The sialyltransferase polypeptide of claim 20, wherein the acceptor molecule is an oligosaccharide, a glycolipid, a glycopeptide, or a glycoprotein.

23. The sialyltransferase polypeptide of claim 20, wherein the sialyltransferase polypeptide further comprises an amino acid tag.

24. The sialyltransferase polypeptide of claim 20, wherein the sialyltransferase polypeptide is fused to an accessory enzyme.

25. The sialyltransferase polypeptide of claim 20, wherein the sialyltransferase polypeptide is selected from the group consisting of the amino acid sequence of FIG. 2 (O:36 amino acid sequence, SEQ ID NO:6) and the amino acid sequence of FIG. 3 (O:19 amino acid sequence, SEQ ID NO:8).

26. An isolated or recombinant sialyltransferase polypeptide;
wherein the sialyltransferase polypeptide transfers sialic acid from a donor substrate to an acceptor substrate; and
wherein the sialyltransferase polypeptide comprises amino acids 1-283 of the amino acid sequence of FIG. 2 (O:36 amino acid sequence, SEQ ID NO:6).

27. The isolated or recombinant sialyltransferase polypeptide of claim 26, wherein the sialyltransferase polypeptide comprises amino acids 1-285 of the amino acid sequence of FIG. 2 (O:36 amino acid sequence, SEQ ID NO:6).

28. An isolated or recombinant sialyltransferase polypeptide;
wherein the sialyltransferase polypeptide transfers sialic acid from a donor substrate to an acceptor substrate; and
wherein the sialyltransferase polypeptide comprises amino acids 1-285 of the amino acid sequence of FIG. 3 (O:19 amino acid sequence, SEQ ID NO:8)

29. The isolated or recombinant sialyltransferase polypeptide of claim 28, wherein the sialyltransferase polypeptide comprises amino acids 1-293 of the amino acid sequence of FIG. 3 (O:19 amino acid sequence, SEQ ID NO:8)

30. An isolated or recombinant nucleic acid that comprises a sialyltransferase polynucleotide sequence that encodes a sialyltransferase polypeptide;
wherein the sialyltransferase polypeptide transfers sialic acid from a donor substrate to an acceptor substrate; and
wherein the sialyltransferase polynucleotide sequence comprises a nucleotide sequence with at least 98% identity to the nucleic acid sequence of FIG. 2 (O:36 nucleic acid sequence, SEQ ID NO:5).

31. The isolated or recombinant nucleic acid of claim 30, wherein the acceptor molecule is an oligosaccharide, a glycolipid, a glycopeptide, or a glycoprotein.

32. The isolated or recombinant nucleic acid of claim 30, wherein the sialyltransferase polypeptide further comprises an amino acid tag.

33. The isolated or recombinant nucleic acid of claim 30, wherein the sialyltransferase polypeptide is fused to an accessory enzyme

34. The isolated or recombinant nucleic acid of claim 30, wherein the sialyltransferase polynucleotide sequence is selected from the group consisting of the nucleic acid sequence of FIG. 2 (O:36 nucleic acid sequence, SEQ ID NO:5) and the nucleic acid sequence of FIG. 3 (O:19 nucleic acid sequence, SEQ ID NO:7).

35. An isolated or recombinant nucleic acid that comprises a sialyltransferase polynucleotide sequence that encodes a sialyltransferase polypeptide;
wherein the sialyltransferase polypeptide transfers sialic acid from a donor substrate to an acceptor substrate; and
wherein the sialyltransferase polynucleotide sequence comprises nucleotides I-849 of FIG. 2 (O:36 nucleic acid sequence, SEQ ID NO:5).

36. An isolated or recombinant nucleic acid that comprises a sialyltransferase polynucleotide sequence that encodes a sialyltransferase polypeptide;
wherein the sialyltransferase polypeptide transfers sialic acid from a donor substrate to an acceptor substrate; and
wherein the sialyltransferase polypeptide comprises nucleotides 1-888 of FIG. 3 (O:19 nucleic acid sequence, SEQ ID NO:7).

37. An isolated or recombinant nucleic acid that comprises a sialyltransferase polynucleotide sequence that encodes a sialyltransferase polypeptide;
wherein the sialyltransferase polypeptide transfers sialic acid from a donor substrate to an acceptor substrate; and
wherein the sialyltransferase polypeptide comprises amino acids 1-285 of the amino acid sequence of FIG. 2 (O:36 amino acid sequence, SEQ ID NO:6).

38. An isolated or recombinant nucleic acid that comprises a sialyltransferase polynucleotide sequence that encodes a sialyltransferase polypeptide;
wherein the sialyltransferase polypeptide transfers sialic acid from a donor substrate to an acceptor substrate; and
wherein the sialyltransferase polypeptide comprises amino acids 1-285 of the amino acid sequence of FIG. 3 (O:19 amino acid sequence, SEQ ID NO:8).

39. An expression vector comprising a nucleic acid sequence of claims 30, 35, 36, 37, or 38.

40. A host cell comprising the expression vector of claim 39.

41. A method of making a sialyltransferase polypeptide, the method comprising growing the host cell of claim 40, under conditions suitable for expression of the sialyltransferase polypeptide.

42. A method of producing a sialylated product saccharide, the method comprising the step of:
a) contacting an acceptor substrate with a donor substrate comprising a sialic acid and a sialyltransferase polypeptide of claims 1, 20, 26, or 28; and
b) allowing transfer of a sialic acid moiety to the acceptor saccharide to occur, thereby producing the sialylated product saccharide
